(19) United States
(12) Patent Application Publication
(10) Pub. No.: US 2018/0014778 A1
(43) Pub. Date: Jan. 18, 2018

(54) ELECTRONICALLY LOCKABLE WEARABLE DEVICE

(71) Applicant: KONINKLIJKE PHILIPS N.V., EINDHOVEN (NL)

(72) Inventors: John Cronin, Bonita Springs, FL (US); Roger Holmes, EINDHOVEN (NL)

(21) Appl. No.: 15/547,857

(22) PCT Filed: Feb. 1, 2016
(86) PCT No.: PCT/IB2016/050489

§ 371 Date: Aug. 1, 2017

Related U.S. Application Data
(60) Provisional application No. 62/111,299, filed on Feb. 3, 2015.

(51) Int. Cl. A61B 5/00; A61B 5/11; G06F 19/00; E05B 73/00; E05B 47/00; A61B 5/01; E05B 47/02; E05B 35/00; G04B 37/14; A61B 5/053

(52) U.S. Cl. CPC A61B 5/681; E05B 73/00; A61B 5/1118; A61B 5/486; A61B 5/4866; A61B 5/7455; G06F 19/3418; A61B 5/0533; A61B 2562/0219; A61B 5/01; E05B 47/026; E05B 2035/009; G04B 37/1486; G06F 19/3481; E05B 47/0004

(57) ABSTRACT

A wearable device includes: a band configured to at least partially surround a body part of a user; a clasp configured to hold the band in a closed position, wherein the band resists removal from the body part while in the closed position; a lock actuator configured to selectively lock the clasp against moving to an open position; a communication interface configured to receive data from at least one other device; and a processor in communication with the lock actuator and the communication interface, wherein the processor is configured to: receive a locking indication that the wearable device is to be unlocked, and in response to receiving the locking indication, signal the lock actuator to allow the clasp to transition to the open position. The device may further comprise a physiological sensor (e.g. an accelerometer for motion sensing), and an indication to allow unlocking of the clasp may for example be received upon reaching a predefined goal measured by said physiological sensor, e.g. after burning a certain amount of a calories.

### CROSS-REFERENCE TO RELATED APPLICATIONS

The present application claims the priority benefit of U.S. provisional application No. 62/111,299 filed Feb. 3, 2015 and entitled “Training Habit Wearable,” the entire disclosure of which is hereby incorporated herein by reference for all purposes.

### TECHNICAL FIELD

Various embodiments described herein relate to wearable devices and more particularly, but not exclusively, to wearable devices that lock against removal from a wearer.

### BACKGROUND

Wearable devices are seeing an increasing rate of adoption across a variety of applications and markets. These devices are attachable in some way to a user (e.g., via a wrist strap, necklace, an adhesive patch, etc.) and leverage this proximity to, attachment to, or even contact with the user to provide new or enhanced functionality. For example, an emergency alert button may be provided by a wearable device so that the wearer is always able to easily call for assistance in the case of a medical emergency. As another example, an accelerometer may be provided by a wearable device to produce data that is useful to estimate the wearer's activity level. As wireless network attachment becomes more feasible for these devices, the potential applications will continue to expand even further.

### SUMMARY

Various embodiments described herein relate to a wearable device (along with a related method and non-transitory machine-readable storage medium) including: a band configured to at least partially surround a body part of a user; a clasp configured to hold the band in a closed position, wherein the band resists removal from the body part while in the closed position; a lock actuator configured to selectively lock the clasp against moving to an open position; a communication interface configured to receive data from at least one other device; and a processor in communication with the lock actuator and the communication interface, wherein the processor is configured to: receive a locking indication that the wearable device is to be unlocked, and in response to receiving the locking indication, signal the lock actuator to allow the clasp to transition to the open position.

Various embodiments are described wherein the locking indication is received via the communication interface.

Various embodiments are described wherein the locking indication is received from a user device of a supporting user other than the user.

Various embodiments additionally include a memory configured to store a locking rule, wherein, in receiving the locking indication that the wearable device is to be unlocked, the processor is configured to evaluate the locking rule against a current context and, based on the evaluation, determine that the wearable device is to be unlocked.

Various embodiments are described wherein, in evaluating the locking rule against a current context, the processor is configured to determine that the locking rule has expired that that, based on the expiration, that the wearable device is to be unlocked.

Various embodiments additionally include a sensor configured to obtain physiological data from the user when the band surrounds the body part, wherein the processor is configured to transmit the physiological data via the communications interface.

Various embodiments additionally include a user interface for outputting communications to the user, wherein the processor is further configured to receive a communication indication that the wearable device is to output a specified communication to the user, and in response to receiving the communication indication, output the specified communication via the user interface.

Various embodiments are described wherein: the user interface includes a vibrator; and the specified communication is at least one vibration.

Various embodiments additionally include a sensor configured to obtain physiological data from the user when the band surrounds the body part, a memory configured to store a communication rule, wherein, in receiving the communication indication that the wearable device is to output a specified communication to the user, the processor is configured to evaluate the communication rule against a current context and, based on the evaluation, the wearable device is to output a specified communication to the user.

Various embodiments additionally include a sensor configured to obtain physiological data from the user when the band surrounds the body part, wherein, in evaluating the communication rule against a current context, the processor is configured to compare the communication rule to the obtained physiological data.

Various embodiments described herein relate to a habit rules engine (along with a related method and non-transitory machine-readable storage medium) including: a communication interface configured to communicate with at least one other device; a memory storing a user profile including an identification of a locking wearable device and a locking rule; and a processor configured to: evaluate the locking rule against a current context to determine whether the locking wearable device is to be unlocked; and in response to determining that the locking wearable device is to be unlocked, transmit a locking instruction to the locking wearable device, wherein the locking instruction instructs the locking wearable device to unlock.

Various embodiments are described wherein, in evaluating the locking rule against the current context, the processor is configured to determine when the locking rule is expired and that, consequently, the locking device is to be unlocked.

Various embodiments are described wherein the processor is further configured to: receive physiological data descriptive of the a user of the locking wearable device; evaluate a goal previously received from the user against the received physiological data; and based on the evaluation, transmit a communication instruction to the locking wearable device instructing the locking wearable device to output a specified communication to the user.

Various embodiments are described wherein the processor is configured to transmit the communication instruction based on the evaluation indicating that the user has failed to meet the goal previously received from the user.

Various embodiments described herein relate to a habit training system including both a locking wearable device and a habit rules engine operating in cooperation with one another.

### BRIEF DESCRIPTION OF THE DRAWINGS

In order to better understand various example embodiments, reference is made to the accompanying drawings, wherein:

FIG. 1 illustrates an example of a system for habit training;

FIG. 2 illustrates an example of a method performed by a locking wearable device;

FIG. 3 illustrates an example of a method performed by a habit rules engine;

FIG. 4 illustrates an example of an environment for habit training;

FIG. 5 illustrates an example of hardware for implementing a locking wearable device;

FIG. 6 illustrates a perspective view of an example of a locking wearable device;

FIG. 7A illustrates a cross-section view of a locking wearable device showing a first example of a clasp and locking actuator;

FIG. 7B illustrates a cross-section view of a locking wearable device showing a second example of a clasp and locking actuator;

FIG. 8 illustrates an example of a method performed by a sensor device for reporting sensor data;

FIG. 9 illustrates an example of a method performed by a locking wearable device for handling instructions from a habit rules engine;

FIG. 10 illustrates an example of a method performed by a locking wearable device for handling early unlock instructions;

FIG. 11 illustrates an example of hardware for implementing various devices that participate in the various systems described herein;

FIG. 12 illustrates an example of a method performed by a parameter identification engine for creating a parameter model;

FIG. 13 illustrates an example of a method performed by a parameter identification engine for updating a parameter model;

FIG. 14 illustrates an example of a method performed by a habit rules engine for evaluating habit rules and remotely controlling a locking wearable device;

FIG. 15 illustrates an example of a method performed by a habit rules engine for auditing habit rules for expiration;

FIG. 16 illustrates an example of a data structure for storing habit rules;

FIG. 17 illustrates an example of a user interface for defining habit rules;

FIG. 18 illustrates an example of a method performed by a habit rules engine for handling an early unlock request; and

FIG. 19 illustrates an example of a user interface for responding to an early unlock request.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., and/or), unless otherwise indicated (e.g., “or else” or “or in the alternative”). Additionally, the various embodiments described herein are not necessarily mutually exclusive and may be combined to produce additional embodiments that incorporate the principles described herein.

While most people have aspirations to adopt good habits or lose bad habits, these goals are usually are often very difficult to attain. After a few days, many people find themselves falling into their old ways. Some people may try to use a reminder to stay on their chosen path such as, for example, a rubber band worn around their wrist. This approach is less than perfect because the person may become accustomed to the rubber band and no longer notice its presence (which thereafter fails to serve as any reminder) or may simply remove the rubber band and fail to put it back on. Accordingly, it would be desirable to provide an improved reminder device to help users adapt their habits as desired such as adopting good habits and dropping bad habits.

According to various embodiments, a wearable device is provided with an electronically-activated clasp that prevents removal of the wearable device from the wearer while in a locked state. In response to the wearer indicating a desire to adopt a habit, the wearable device locks and does not unlock until a requested amount of time has passed, thereby serving as at least a static reminder to work toward the habit for the specified time period. In some embodiments, the wearable device also provides dynamic reminders to participate in activities related to the habit. For example, if the wearer has indicated a desire to adopt a habit of burning 2000 calories in a day, a sensor in the wearable device may be used to estimate energy expenditure and, if the wearer is not on track to meet the daily goal, vibrate to inform the wearer that they should take some action to increase their energy expenditure for the day. Thus, the locking wearable device may provide intelligent reminders at opportune times to assist in habit formation. Various additional and alternative functionality will be apparent in view of the following description.

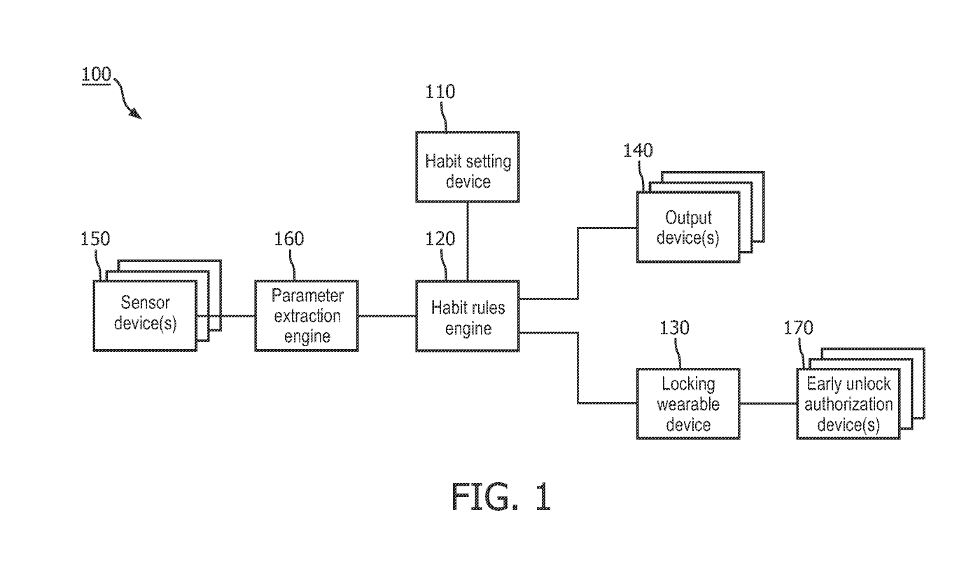

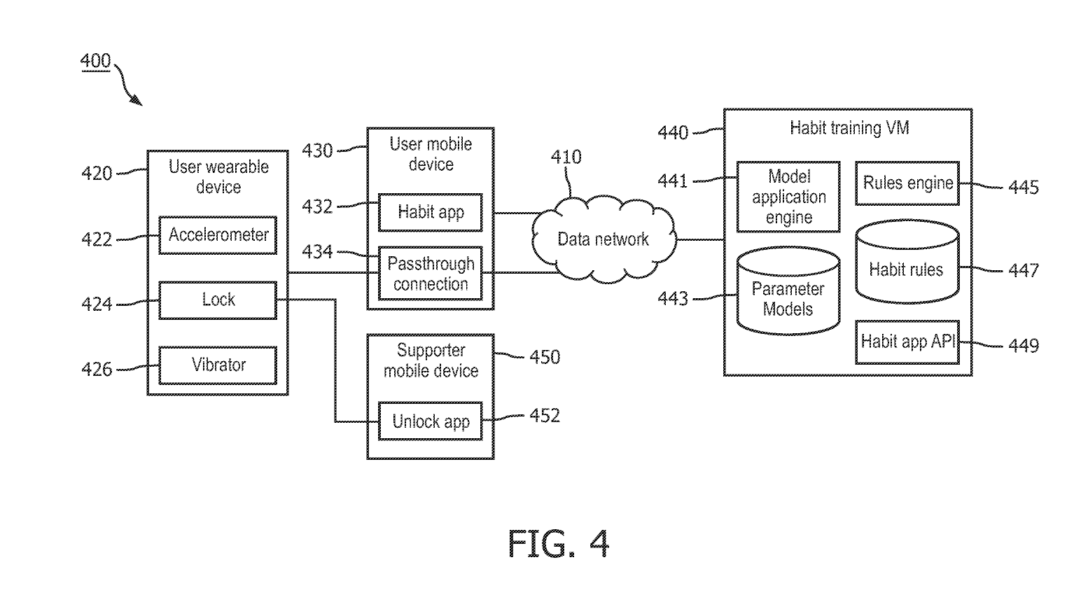

FIG. 1 illustrates an example of a system 100 for habit training. The system 100 shown illustrates various functional components and some interactions therebetween. It will be appreciated that such functional components will be implemented using physical hardware and, in some embodiments, software executed by the hardware. Thus, each functional device or engine may be embodied in a dedicated hardware device. Additionally, in some embodiments, two or more of the functional devices of the system 100 may be embodied in a single hardware device. For example, the sensor devices 150, locking wearable device 130, and output devices 140 may all belong to a single wearable device worn around, for example, the wrist of a wearer. An example of one hardware instantiation of the system 100 will be described in greater detail below with respect to FIG. 4.

While the system 100 shows some functional devices as being single devices and others as including multiple similar devices, it will be understood that alternative arrangements are possible. For example, an alternative system may use only a single sensor device 150 but may include multiple redundant habit rules engines 120 (e.g., with a load balancer, not shown, to distribute requests, user data, or other actionable information evenly therebetween to enable the habit rules engines to serve a large number of wearers and locking wearable devices 130).

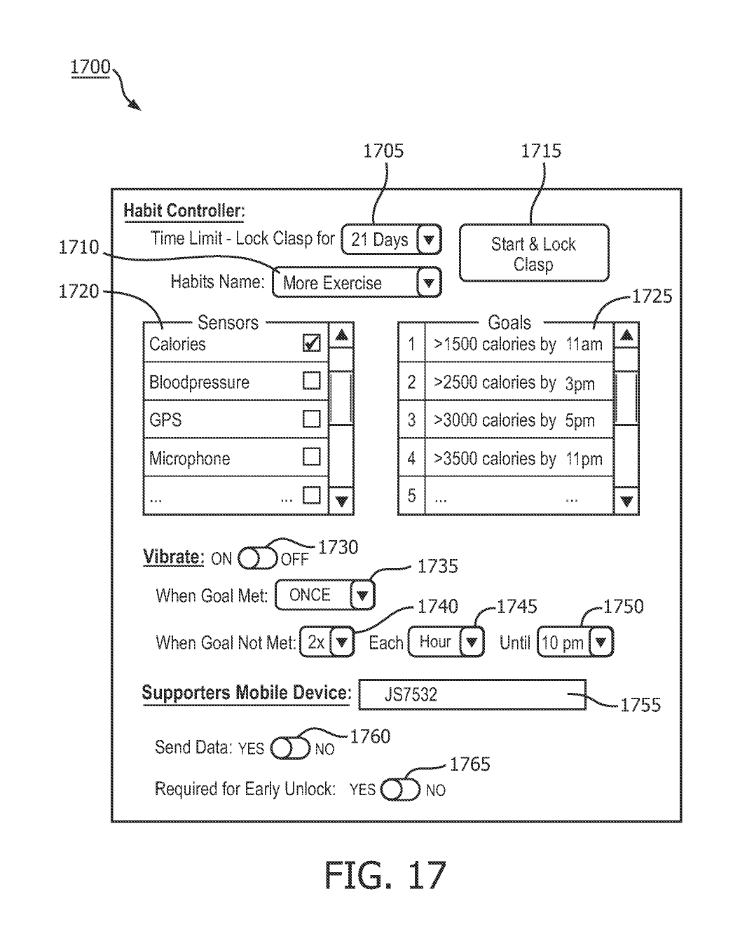

The operation of the system begins with a wearer operating a goal setting device 110 to identify one or more habits that the user wishes to adopt or lose (collectively, habits to “change”). In various embodiments, the habit setting device 110 may be a wearable device (e.g., the locking wearable device 130), a mobile device, tablet, personal computer or other device capable of receiving indications of desired habit changes. In some embodiments, such as those where the habit setting device 110 and habit rules engine 120 are collocated in a single hardware device, the habit setting device 110 may store the desired habit changes (e.g., as a set of rules related to the habit or associated periodic goals) locally. In other embodiments, the habit setting device 110 may transmit the desired habit changes to the habit rules engine 120 (e.g. via an API accessed by a habit app or via a habit web server accessed by a web browser and run by or in association with the habit rules engine 120). An example interface provided via a habit setting device will be explained in greater detail below with respect to FIG. 17.

In various embodiments, a desired habit change is expressed in the habit rules engine as one or more “habit rules.” The habit rules may serve two (or more) separate purposes: defining when the locking wearable device 130 should be unlocked (and, therefore, may also constitute “locking rules”) and defining when and what communications should be sent to the output devices 140 (and, therefore, may also constitute “communication rules”). As such, a habit rules engine may constitute a “locking rules engine” when applying locking rules and a “communication rules engine” when applying communication rules. In some embodiments habit rules may accomplish only one of these functions (e.g., in embodiments wherein no locking is performed or where no communications are sent), while in other embodiments separate locking rules and communication rules are defined so that no single habit rule accomplishes both functions. In some embodiments the habit rules engine 120 may be split between multiple devices. For example, a habit rules engine 120 implemented at a VM or in a output device 140 may handle communication rules (or communication rule aspects of habit rules impacting both communication and locking) while a habit rules engine 120 implemented in the locking wearable device 130 may handle locking rules (or locking rule aspects of habit rules impacting both communication and locking).

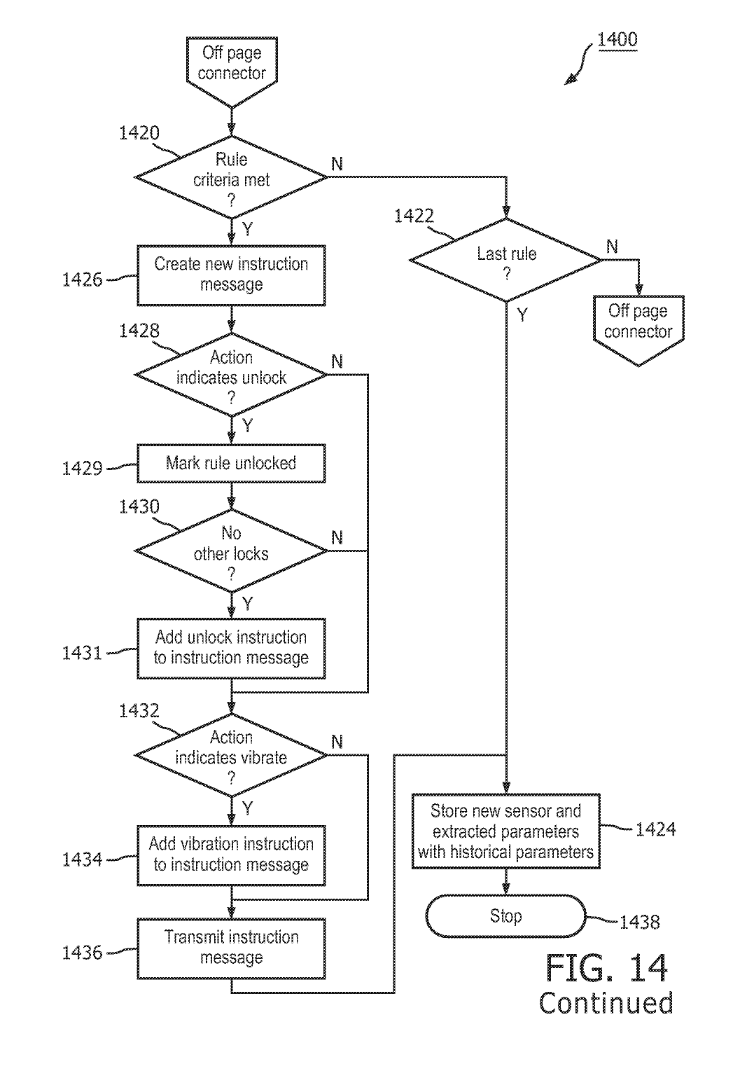

The habit rules engine 120 may be a wearable device (e.g., the locking wearable device 130), a mobile device, tablet, personal computer, server, virtual machine, or other device capable of evaluating habit rules. As will be explained in greater detail below via various examples with respect to FIGS. 14-16, the habit rules engine 120 may periodically (e.g., at scheduled times or upon request by another process or device) evaluate habit rules against a current context to determine whether any actions should be taken such as unlocking a wearable device or effecting communication via the output devices 140. This evaluation against a current context may involve comparing habit rule criteria against sensor data or parameters extracted therefrom by the parameter extraction engine 160 to determine whether an associated action should be performed. Additionally or alternatively, the evaluation may include comparing a current date or time against an expiration date of the rule to determine whether an expiration action (e.g., unlocking the locking wearable device 130) should be performed. Where the evaluation results in an action to be performed by a device that is not collocated with the habit rules engine 120, the habit rules engine 120 may transmit one or more instructions to the remote device to effect the performance of the action.

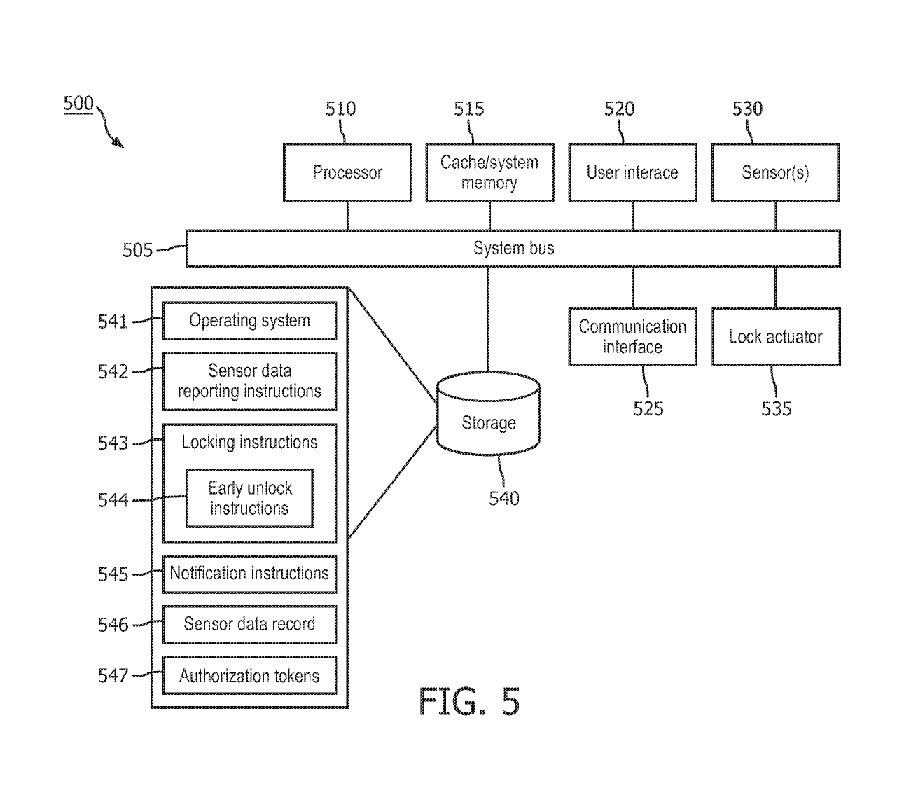

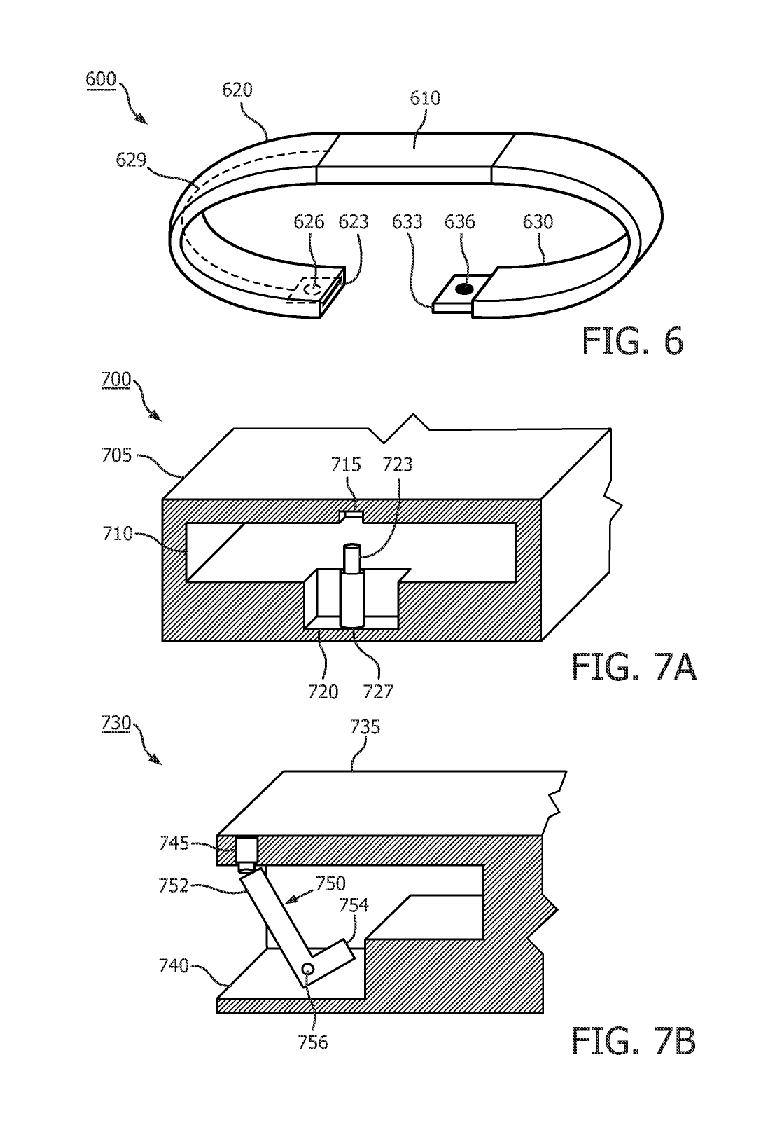

The locking wearable device 130 may be any device that may be attached to a wearer and locked against removal. For example, that locking wearable device may take the form of a wristwatch, having a band that extends around the wearer's wrist and is held by a clasp. During normal operation (i.e., after the wearer has submitted or activated a desired habit change via the habit setting device 110), the locking wearable device is attached to the user and the clasp is locked against being opened. Upon receiving an instruction from the habit rules engine, the locking wearable device 130 “unlocks” and allows the clasp to be moved to the open position, either by actively moves the clasp to the open position or by no longer impeding the clasp against being manually moved to the open position. Example embodiments of locking wearable devices will be described in greater detail below with respect to FIGS. 5-6.

In some alternative embodiments, the locking wearable device 130 may be mounted over another device (e.g., a wearable sensor device) and prevent removal of that other device according to the methods and systems described herein. For example, the locking wearable device 130 may surround, block, or otherwise prevent operation a standard watch clasp to prevent access for unlocking. As such, in this example, the locking wearable device 130 may be usable in conjunction with any watch and would not require the wearer to adopt the particular wearable device incorporating the locking feature which may be undesirable if the user already wears a device that they prefer not to replace or for other fashion reasons. In some such embodiments, the locking wearable device 130 may be substantially hidden from view when locked to the other worn device. Where the other worn device includes sensors, the other device and locking wearable device 130 may communicate with each other to share, e.g., physiological data or locking instructions via a short range communication protocol such as NFC or Bluetooth. Upon unlocking, the locking wearable device 130 of these embodiments may fall off or otherwise be removable to permit access to or operation of the watch (or other worn device) clasp (or other closure element).

In some embodiments, a longer or shorter band may be provided to enable the locking wearable device 130 to be worn around other parts of the body (e.g., ankle, leg, arm, waist, finger, etc.). In some embodiments, the band is adjustable (at least in a worn configuration) to accommodate different body parts or different anatomies. As used herein, the term “band” will be understood to encompass virtually any material that is sufficiently long and flexible to substantially surround a body part of the wearer, meeting the other end of the wearable device (e.g., another band portion or a central electronic hub) and includes (non-exclusively) leather, fabric, and metal watchbands; belts; chains; necklaces (e.g., choker style necklaces); or an elastic or other material strap. In some embodiments, the band may be selectively openable (e.g., like a typical watch) while, in other embodiments that band may be permanently closed (e.g., like a typical ring) yet lockable (e.g., through an expandable inner diameter that, when expanded, resists removal). It will be apparent that, in some embodiments, a locking wearable device may include attachment means in alternative or addition to a band such as, for example, adhesive (e.g., in the case of a wearable patch or bandage), a post (e.g., in the case of a wearable to be worn as a piercing), an article of clothing (e.g., in the case where electronics are incorporated into a garment), etc.

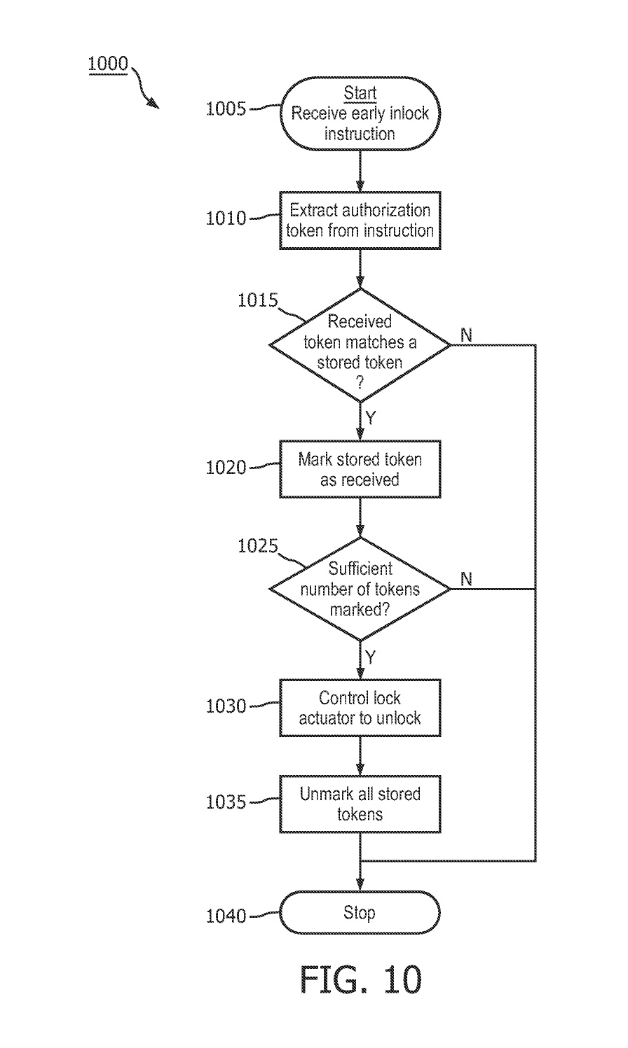

As used herein, the term “clasp” will be understood to encompass more than merely the strap buckle examples described herein. The term “clasp” will be understood to encompass all “band clasps” including “watch clasps” (e.g., strap buckle clasps, deployment clasps, and jewelry-style watch clasps) and “jewelry clasps” (spring ring, lobster claw, bayonet, barrel, open box, FIG. 8, toggle, s-hook, mystery, magnetic, pearl, or bracelet catch clasps). Further, the unqualified term “clasp” will be understood to encompass virtually any device that mechanically resists removal of a wearable device (whether held by a band or other means) while in a “closed position.”

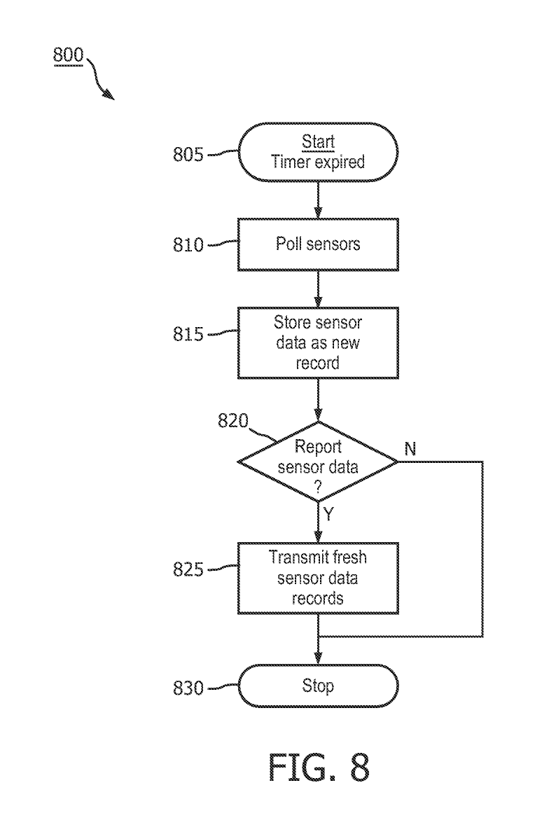

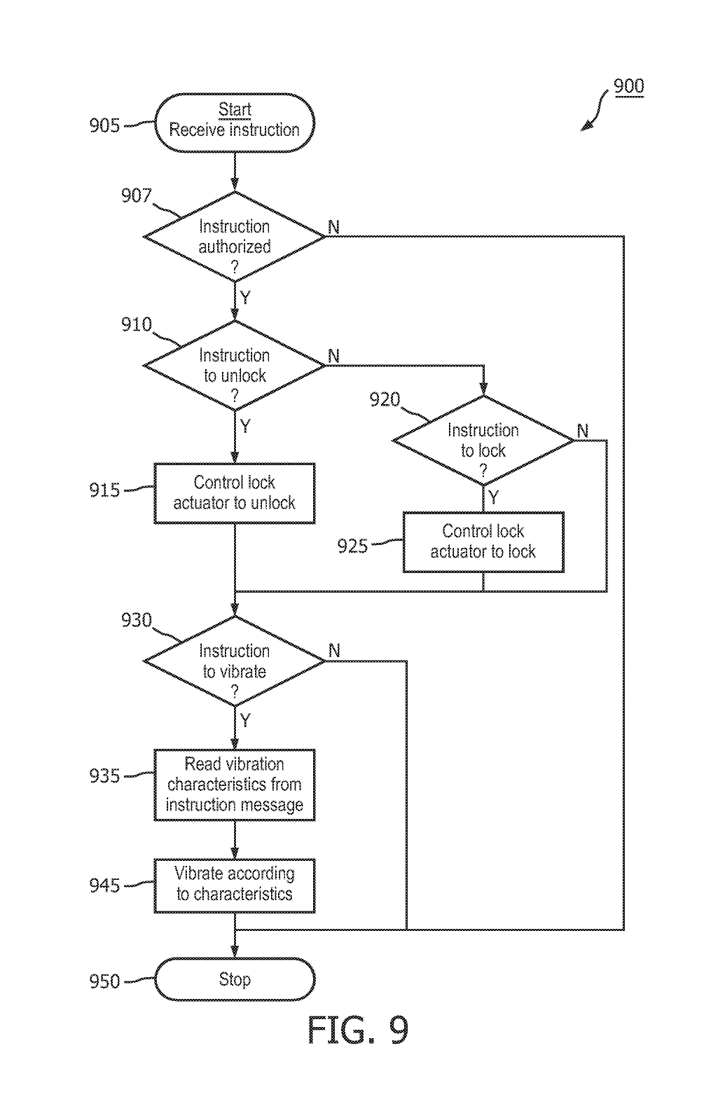

In various embodiments, the locking wearable device 130 also includes an electronically-operated locking actuator that, when actuated enables movement of the clasp to the open position or otherwise enables removal of the locking wearable device from the user. For example, the locking actuator may be an electro-magnet, a solenoid, or other device capable of receiving an electronic signal and effecting or allowing mechanical movement of the clasp to an open position. In some embodiments, the locking actuator may, upon receiving a signal, actively move the clasp to the open position while, in other embodiments, the locking actuator may actively move another component that previously inhibited manual movement of the clasp to the open position. In other embodiments, the locking actuator may cease acting on a physical component upon receiving the signal; thereafter, the physical component may move freely or automatically move out of engagement (e.g., through operation of gravity) to cause or enable manual movement of the clasp to the open position. Example embodiments of a clasp and locking actuator will be described in greater detail below with respect to FIGS. 7A-B.

The output device(s) 140 may be virtually any device capable of outputting a communication to the wearer (or other interested party) such as a mobile device, tablet, personal computer, or a wearable device (e.g., the locking wearable device 130). Upon receiving a communication instruction from the habit rules engine 120, the output device 140 outputs one or more communications defined by the communication instruction. For example, in various embodiments, the output device 140 includes a vibrator (e.g., as part of the locking wearable device 130). The communication instruction may instruct the output device 140 to vibrate as a communication to the wearer or otherwise holder of the output device 140. In some embodiments, the communication instruction may include vibration characteristics such as, for example, number of vibrations, length of vibrations, interval between successive vibrations, or period between each group of vibrations. As another example, the output device 140 may include a display device (e.g., as part of a mobile device or tablet) capable of displaying a message to the wearer or other interested party. In such embodiments, the communication instruction may include or otherwise specify (e.g., by reference to a network resource or resource that is local to the output device 140) a textual, image, or video message to be output to the user. Various additional types of visual, audio, tactile, or other outputs along with appropriate output devices 140 for communicating the outputs will be apparent. In some embodiments, such as those where the output device is implemented in a mobile device or tablet, the communication instruction may be received and executed by a habit training app that was previously installed on the output device 140 for communication with the habit rules engine 120.

As noted above, in various embodiments habit rules may be evaluated against sensor data or parameters derived therefrom. Accordingly, in some embodiments, one or more sensor devices 15 are provided for collecting physiological data about the wearer. In some such embodiments, one or more of the sensor devices 150 may be implemented as part of the locking wearable device 130 or output devices 140. Various sensors useful for tracking performance, progress, or other statistics related to habits or associated goals will be apparent. For example, the sensor devices 150 may include accelerometers, conductance sensors, optical sensors, temperature sensors, microphones, cameras, etc. These or other sensors may be useful for sensing, computing, estimating, or otherwise acquiring physiological parameters descriptive of the wearer such as, for example, steps taken, walking/running distance, standing hours, heart rate, respiratory rate, blood pressure, stress level, body temperature, calories burned, resting energy expenditure, active energy expenditure, height, weight, sleep metrics, other habit-specific parameters such as hours of music practice, etc.

In various embodiments, the sensor devices 150 may periodically transmit obtained sensor data or other parameters to other devices for further use. For example, the sensor devices 150 may periodically transmit gathered data directly to the parameter extraction engine 160 or to a wearable device management framework (not shown) such as the AWS Internet of Things (IoT) cloud platform, which may later be polled by the parameter extraction engine 160.

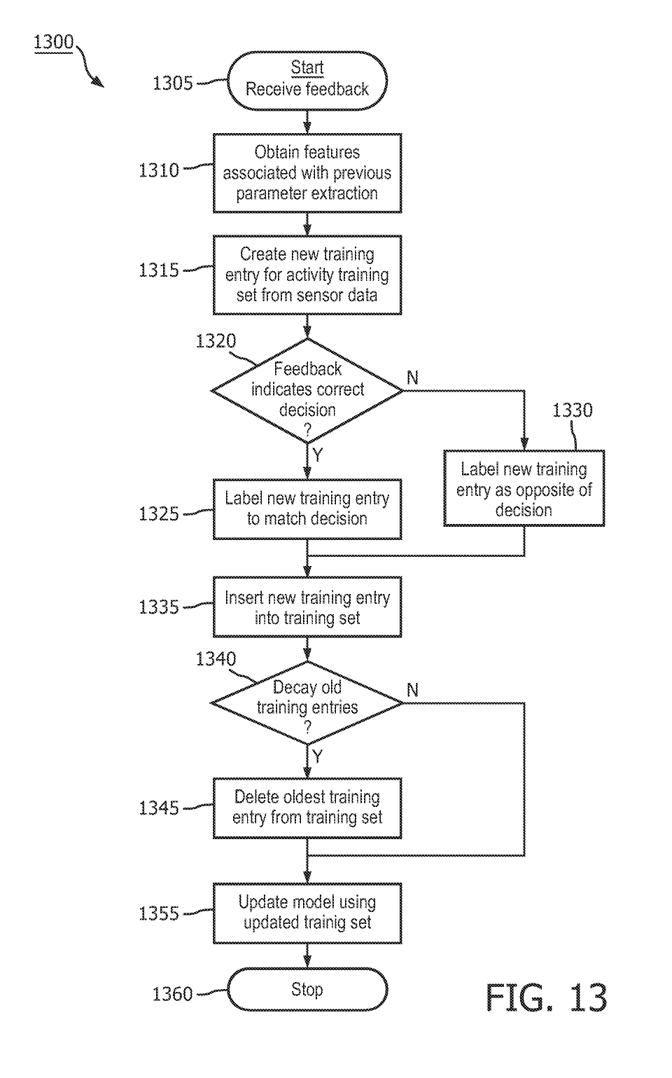

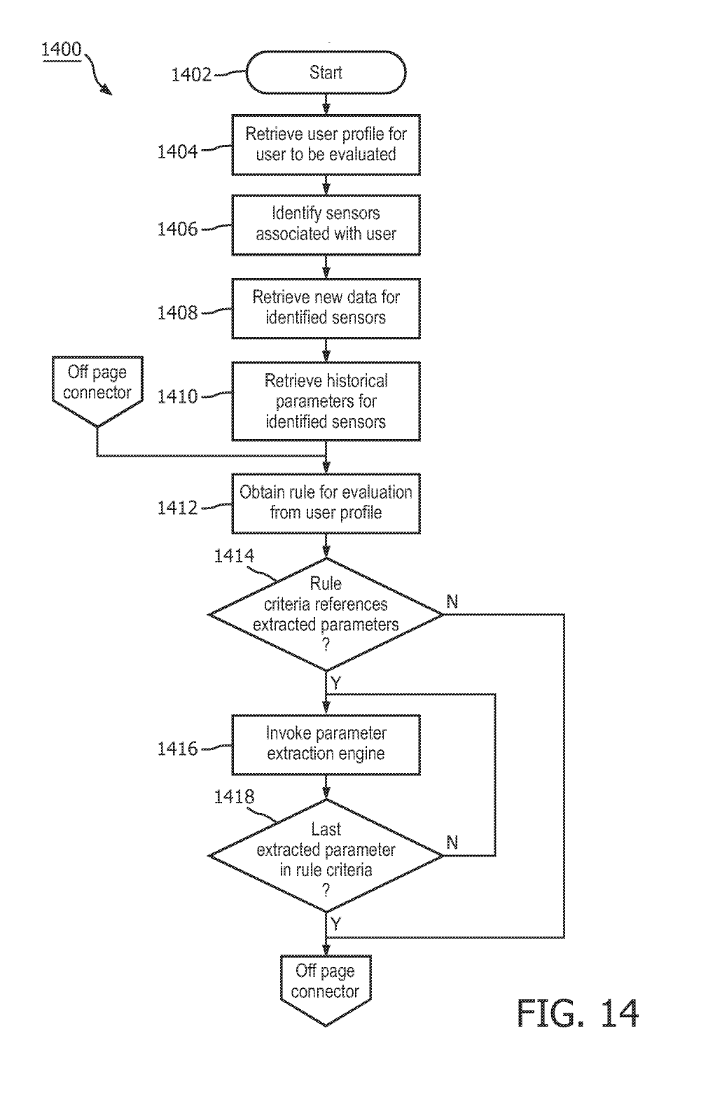

While some physiological parameters useful for evaluating habit rules may be obtained directly from the sensors 150, others are obtained by “extracting” them from other available data (including the sensor data). As such, a parameter extraction engine 160 processes the available data to compute, discern, or otherwise extract additional parameters for use by the habit rules engine 120 in evaluating habit rules. The parameter extraction engine 160 may be a wearable device (e.g., the locking wearable device 130), a mobile device, tablet, personal computer, server, virtual machine, or other device capable of processing data as described herein and, in some embodiments, may be collocated on the same hardware as the habit rules engine 120. Some parameters may be calculated by a specific algorithms for computing the parameter. For example, an algorithm may be defined to extract the average heart rate over the past 7 days. Other parameters may be calculated according to a mathematical formula, such as a formula generated according to a machine learning approach such as regression, neural networks, or Bayesian networks. For example, calories expended may be extracted by inputting accumulated accelerometer data into a formula generated using linear regression. Example methods for generating and maintaining such learned “parameter models” will be described in greater detail below with respect to FIGS. 12-13.

In some embodiments, the locking wearable device 130 may provide one or more methods of unlocking or otherwise removing the locking wearable device from the wearer prior to application of a habit rule by the habit rules engine 120 indicates that the locking wearable device 130 is to be unlocked. For example, in some embodiments, the locking wearable device 130 may include a frangible element, a strap formed of cuttable material, or an otherwise destructible component to enable the locking wearable device to be removed while destroying at least a portion of the device, thereby discouraging early removal but enabling removal in an emergency situation. In some embodiments, the destructible component may be reparable at a relatively high effort level, skill level, or cost.

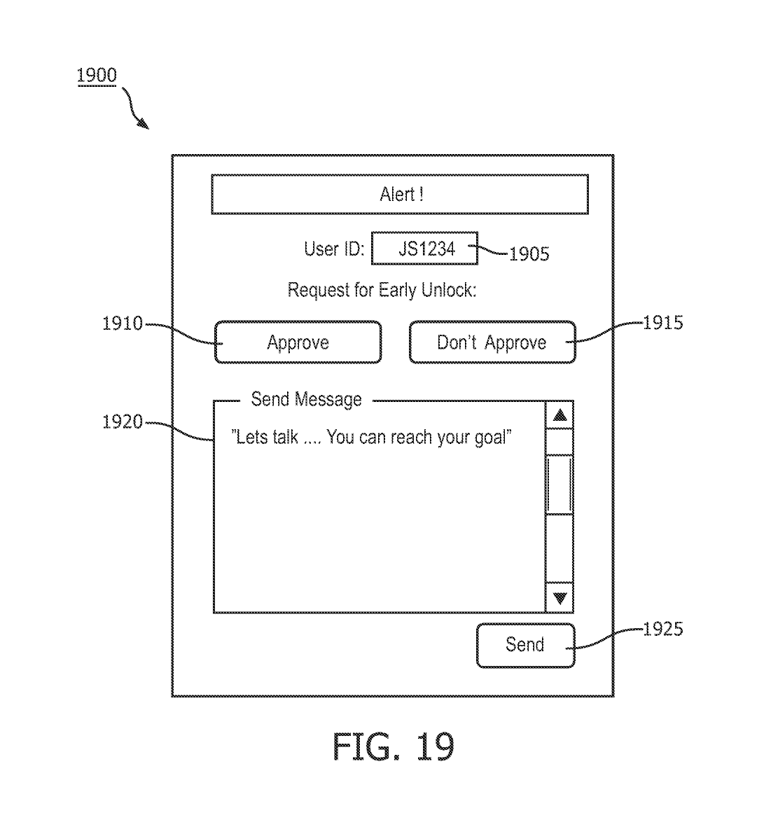

In other embodiments, the locking wearable device 130 may receive and execute instructions for early unlocking by operating the locking actuator. For example, in some embodiments, upon identifying a desired habit change via the habit setting device 110, the wearer may also indicate one or more early unlock authorization devices 170. The locking wearable device 130 may then effect an early unlocking only upon receiving an instruction to do so from one, more, or all of the identified early unlock authorization devices 170. The early unlock authorization devices 170 may be a wearable device (e.g., the locking wearable device 130), a mobile device, tablet, personal computer, or any other device capable of transmitting early unlock instructions based on user input. In some embodiments, such as those where the early unlock authorization device 170 is a mobile device or tablet, an early unlock app may provide an interface for receiving user approval or denial of requests for early unlock. In this way, the wearer may identify one or more supporters that are able to authorize and early unlock of the locking wearable device and, thereby, receive a notification when the user indicates a desire to give up on their desired habit change. An example of a user interface for approving or denying early unlock will be described in greater detail below with respect to FIG. 19.

Various additional features and modifications to the locking wearable device 130 will be apparent. In some embodiments, the locking wearable device 130 ay be waterproof to protect sensitive electronics, enabling the user to wear the locking wearable device 130 at all times, including while bathing, swimming, washing hands, etc. In some embodiments, the locking wearable device 130 may be adjustable for comfort. For example, in some embodiments, a band may be capable of adjustment in both directions (loosening and tightening) while unlocked and only in one direction (e.g., tightening) while locked. In other embodiments, the band may be adjustable in both directions while locked, but only able to be loosened enough for comfort without enabling the locking wearable device 130 to be removed. For example, upon closing or locking the locking wearable device 130, the current setting may be set as the maximum looseness allowed; thereafter, the user may tighten the locking wearable device 130 and subsequently loosen the locking wearable device 130 but only to the extent of the originally-set position upon closing or locking. Alternatively, the locking wearable device 130 may allow loosening to a predetermined setting or to a predetermined difference (e.g., number of ratchet clicks) beyond the initial setting. Various mechanisms for allowing and limiting adjustment of the clasp without opening the clasp or for providing other adjustment mechanisms separate from the clasp on the band (or other user attachment element such as a necklace or belt) of the locking wearable device 130 will be apparent.

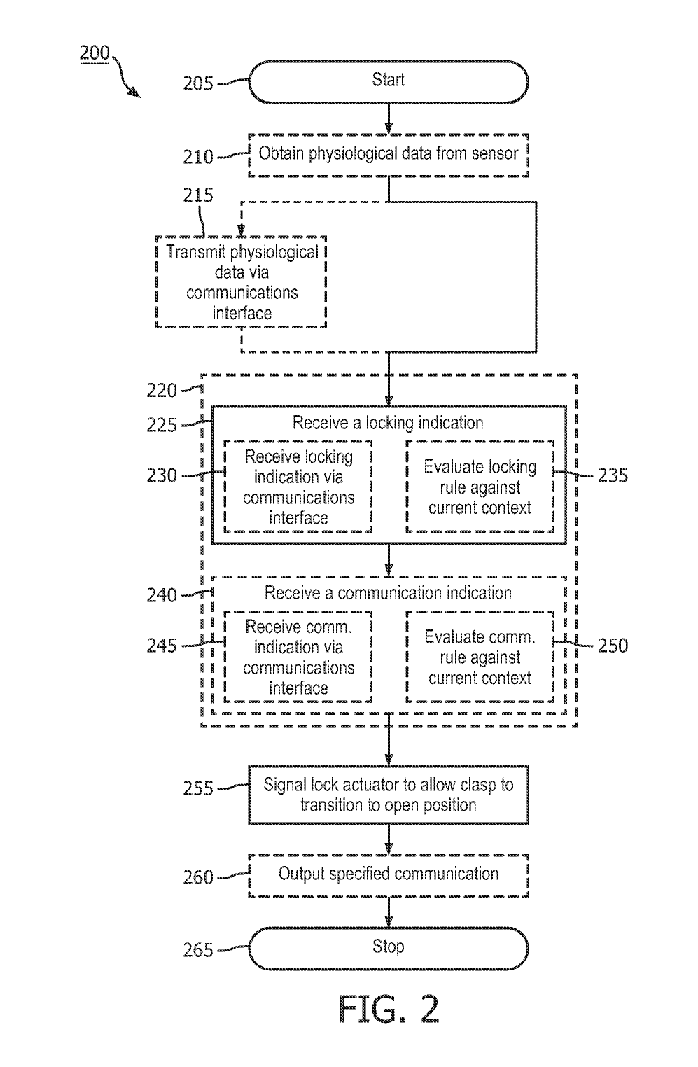

FIG. 2 illustrates an example of a method 200 performed by a locking wearable device such as the locking wearable device 130 of the example system 100. It will be noted that while various steps are shown in dashed form to indicate that they are optional, the use of solid boxes does not imply that the steps are necessary, indispensable or unalterable. Instead, the solid boxes illustrate various central steps according to some embodiments while, in other embodiments, the same solid boxes could be displayed as optional, dashed boxes. Further, while dashed box 215 is shown as being circumvented by one path, this does not imply that this step is “more optional” than other optional steps; instead, box 215 is aligned for illustrative purposes, showing that this step is particularly useful in embodiments implementing step 230 or step 245, with which step 215 is aligned.

The method begins in step 205 and proceeds to step 210 where the wearable device obtains physiological data from one or more sensors (e.g., in embodiments where sensors are integrated into the locking wearable device or embodiments where the locking wearable device communicates with nearby external sensor devices directly or via a wearable device management framework). In some embodiments where the physiological data is processed by an external device (e.g., an external parameter extraction engine or habit rules engine), the locking wearable device transmits the physiological data to the external device in step 215.

Next, in step 225, the locking wearable device receives a locking indication indicating that the locking wearable device is to be unlocked. As will be apparent in view of the present and following description, the locking wearable device may receive such a locking indication in multiple different manners. For example, where the habit rules engine or early unlock authorization device is external to the locking wearable device, the locking wearable device may receive the locking indication via a communications interface from said external device in step 230. In other embodiments, the locking wearable device may include the habit rules engine and, as such, may evaluate the locking rule against the current context in step 235 to determine that the locking wearable device is to be unlocked.

In embodiments where the locking wearable device also includes an output device (e.g., a vibrator), the locking wearable device may also receive a communication indication 240 in step 240. As with the locking indication, the locking wearable device may receive such a communication indication in multiple different manners. For example, where the habit rules engine or early unlock authorization device is external to the locking wearable device, the locking wearable device may receive the locking wearable device indication via a communications interface from said external device in step 245. In other embodiments, the locking wearable device may include the habit rules engine and, as such, may evaluate the communication rule against the current context in step 250 to determine a communication to be output to the user.

In some embodiments, the steps of receiving a locking indication 225 and receiving a communication indications 240 may be accomplished at the same time as part of a single step 220. For example, in some embodiments, the two indications may be received from an external device as part of a single instruction message. As another example, in some embodiments, steps 235 and 250 may constitute a single step of evaluating habit rules against a current context, the outcome of which may indicate both locking actions and communications.

In step 255, based on receiving the locking indication in step 225, the locking wearable device signals its lock actuator to allow the clasp to transition to the open position (e.g., by actively opening the clasp or ceasing to prevent manual opening of the clasp), thereby allowing the wearable device to be removed. Further, where the locking wearable device receives a communication indication in step 240, the locking wearable device outputs the communication specified thereby (e.g., vibrations). The method 200 then proceeds to end in step 265.

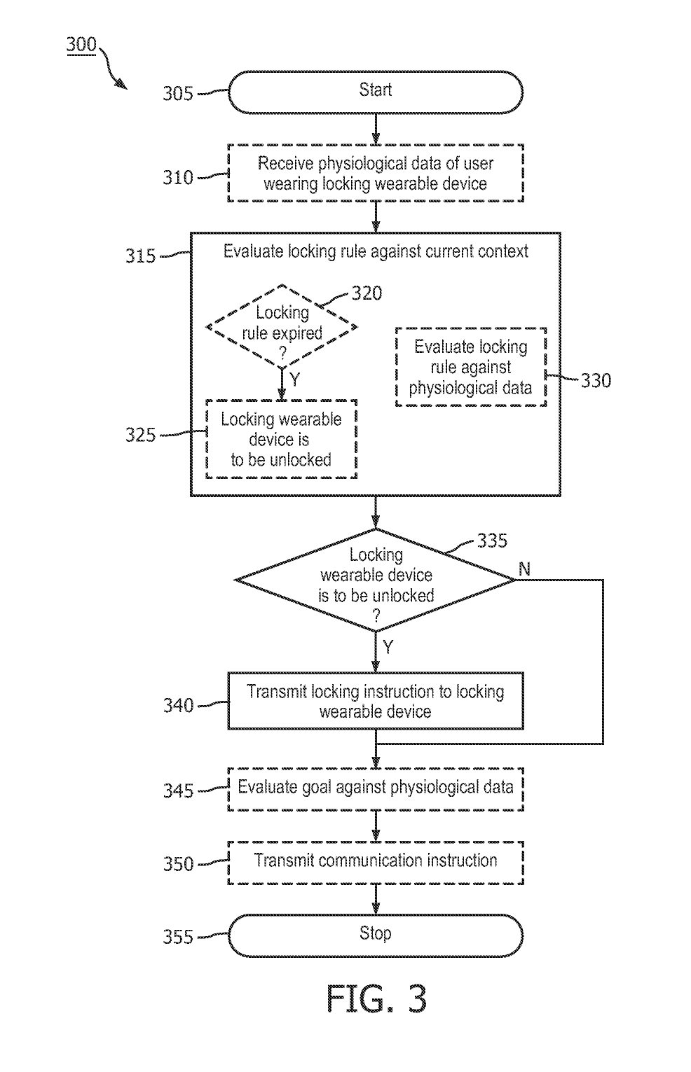

FIG. 3 illustrates an example of a method 300 performed by a habit rules engine. It will be noted that while various steps are shown in dashed form to indicate that they are optional, the use of solid boxes does not imply that the steps are necessary, indispensable or unalterable. Instead, the solid boxes illustrate various central steps according to some embodiments while, in other embodiments, the same solid boxes could be displayed as optional, dashed boxes. While the method 300 is described as performed by a habit rules engine that is separate from a locking wearable device, various modifications will be apparent to implement the method 300 in a habit rules engine that is collocated on the same hardware as the locking wearable device.

The method 00 begins in step 305 and proceeds to step 310 where the habit rules engine receives physiological data of a user wearing a locking wearable device. For example, the habit rules engine may receive sensor data from one or more sensors or other parameters extracted therefrom by a parameter extraction engine (which, in some embodiments, may be collocated on the same hardware as the habit rules engine). Next, in step 315, the habit rules engine evaluates a rule against a current context. For example, the habit rules engine may determine whether a locking rule is expired in step 320 and, if so, determine that the locking wearable device is to be unlocked in step 325. As another example, the habit rules engine may evaluate a locking rule against the physiological data obtained in step 310 and, based on determining that the rule is applicable, determine that the rule indicates that the locking wearable device is to be unlocked. It will be apparent that similar steps 320, 325, 330 may be performed as part of the locking wearable device's rule evaluations 235, 250 in method 200. If it is determined in step 335, that step 315 has determined that the locking wearable device is to be unlocked, then the method 300 proceeds to step 340 where the habit rules engine transmits a locking instruction to the locking wearable device to effect the unlocking.

In step 345, the habit rules engine evaluates one or more goals (e.g., subgoals associated with the desired habit change or achieving the habit change itself) to determine whether any communication should be sent. In some embodiments, this may involve evaluating one or more communication rule. In some such embodiments, evaluation of the communication rule may occur at the same time as evaluation of the locking rule 330 e.g., as an evaluation of all habit rules). Next, in step 350, the habit rules engine transmits communication instructions to output devices (such as the locking wearable device in some embodiments) based on the evaluation in step 345. In some embodiments, the locking instruction 340 and communication instructions 350 may be transmitted as part of the same instruction message. The method 300 then proceeds to end in step 355.

FIG. 4 illustrates an example of an environment 400 for habit training. The environment 400 may include an example implementation of the example system 100 described above. For example, the user wearable device 420 may implement a locking wearable device 130, output device 140, and sensor device 150; the user mobile device may implement a habit setting device 110 and output device 140; the habit training VM 440 may implement a parameter extraction engine 160 and habit rules engine 120; and the supporter mobile device 450 may implement an early unlock authorization device 170. The example environment 400 will be described in terms of a system useful for helping a wearer obtain a habit of burning 200 calories a day; various modifications (e.g., additional/alternative sensors, parameter models, and habit rules) for assisting in other habit changes will be apparent.

As shown, a data network 410 interconnects various devices 410, 430, 440, 450 of the environment 400. The data network 410 may be virtually any device or group thereof for facilitating data communications. As such, the data network 410 may include a LAN, WAN, carrier network (3G/LTE/4G/etc.), or the Internet.

The user wearable device 420 (example hardware for which will be described in greater detail below with respect to FIG. 5) includes an accelerometer 422 for gathering motion data of the wearer (which may be useful for estimating calorie expenditure), an electronically actuated lock 424 for selectively locking the user wearable device 420 to the wearer, and a vibrator for outputting communications to the user.

The user mobile device 430 and supporter mobile device 450 may be mobile phones that communicate either directly or indirectly (e.g., via the habit training VM 440) with the user wearable device 420. The user mobile device 430 includes a habit app 432 for interfacing with the habit training VM (e.g., for allowing the wearer to indicate desired habit changes) and a passthrough connection 434 (which may be implemented as part of the habit app 432) for facilitating communication between the user wearable device 420 and other devices attached to the data network 410 such as the habit training VM 440. For example, the user wearable device may communicate with the passthrough connections 434 via an NFC, Bluetooth, WiFi, or other wireless or wired connection; the passthrough connection may then communicate with the data network 410 on the user wearable device's 420 behalf. In other embodiments, the user wearable device 420 may instead connect directly to the data network 410 without any facilitation by the user mobile device 430. The supporter mobile device 450 includes an unlock app 452 for transmitting early unlock instructions to the lock 424 of the user wearable device 420.

The habit training VM 440 is a virtual machine that may be provisioned in a cloud computing architecture, though it will be apparent that standard, non-cloud servers may be used in other embodiments. The habit training VM 440 includes a model application engine 441 for applying one or more trained parameter models 443 to received sensor data to extract new parameters for use by the rules engine 445. The rules engine 445 evaluates habit rules 447 to determine when instructions to unlock and output communications should be transmitted to the user wearable device 420 or user mobile device 430. A habit app API 449 enables the habit app 432 to configure the habit rules 447 to be evaluated by the rules engine 445.

Having described the example components of the example environment 400, an example of the operation of this environment will now be described. Various modifications to this operation will be apparent in view of the present disclosure.

A wearer begins by downloading and accessing the habit app 432 on their mobile device 430. Using an interface provided by the habit app 432, the wearer indicates that, over the next 30 days, the wearer would like to burn 2000 calories a day and that, if this goal is not reached by 6 pm each day, the wearer should be notified via 3 short vibrations on the wearable device and a message to the user mobile device indicating the day's total calorie expenditure so far. The wearer also indicates that the wearer should be notified each day when the 200 calorie goal is attained via one long vibration. The habit app 432 transmits this input to the habit app API 449, which creates two new rules and stores them with the other habit rules 447 for this or other users. The user then puts on the user wearable device 420 which, by virtue of activation of the rules via the habit app (e.g., by receiving an instruction to lock from the user mobile device 430, the habit training VM 440, or simply by default), the lock engages preventing or otherwise resisting removal of the user wearable device from the wearer.

As the wearer goes about the day, the accelerometer 422 gathers motion data and transmits it, via the passthrough connection, to the habit training VM. The model application engine 441 process the motion data in accordance with a calorie expenditure model 443 to estimate the current day's calorie expenditure and persists this data for later use by the rules engine 445. Later, at 6 pm, the rules engine evaluates the habit rules 447 and, based on the model applications engine's 441 most recent calorie expenditure estimation on 1500 calories, transmits an instruction to the user wearable device 420 to vibrate three short times and an instruction to the habit app 432 of the user mobile device 430 (or, alternatively, via an SMS message, email, or other communication medium) to display the message “You have only burned 1500 calories today! Now that the children are in bed, maybe you should go for a run.”

Receiving this message, the wearer goes for a run as the accelerometer continues to transmit motion data to the habit training VM 440 which continues to update its estimated energy expenditure. In response to an update of the energy expenditure parameter as the user is running, the rules engine 445 is invoked and, evaluating the rules, sees that the new energy expenditure parameter of 2024 calories exceeds the 2000 calorie threshold and transmits and instruction to the user wearable device 420 to output one long vibration. The user, recognizing this output to indicate that a daily goal has been achieved, ends their run.

This general operation continues until the 30th day, when the rules engine again attempts to evaluate the two rules. Seeing that these rules have passed expiration, transmits an instruction to the user wearable device to unlock, thereby allowing the wearer to remove the user wearable device. Having been gently reminded each day for 30 days to reach the 2000 calorie goal, the user has obtained a healthy exercise routine and will no longer need reminders to continue to reach this 2000 calorie goal.

As will be understood, various embodiments may differ from this example in various respects. For example, in some embodiments or for some goals, the end-goal may not be so statically tied to periodic goals as in the example here where the end goal is to burn 2000 calories a day and the daily goals are therefore simply to burn 2000 calories. In some embodiments, selection of a particular end goal (e.g., burn 2000 calories a day, lose 10 pounds, practice music more, etc.), the system may automatically select or suggest a set of periodic goals that will tend to help the user achieve the end goal. For example, in various embodiments, the system includes a database prepopulated with end goals for selection and associated periodic goals to present to the user when the end-goal is selected.

In some embodiments, these periodic goals may be, at least in part, calculated and adapted to the particular user. Periodic goals may contain goals for any period of time and be flexible to known daily variations of habitual behavior. For example, to capture past data based insights that said user would always engage in vigorous activity on Tuesday evenings hence the day target for Tuesdays would be adjusted to a higher value to include the evening activity (rather than using a daily goal that is lower than the habitual level prior to using the system).

Thus, various approaches may provide varying degrees of flexibility in goal setting. Some embodiments may utilize separate goal setting logic executed by a processor that may provide goal setting that is (without limitation) static and not personalized; static based on behavior assessment (i.e. personalized); dynamic and not personalized; dynamic based on one-time behavior assessment; dynamic based on continuous repetitive behavior assessment; or dynamic based on algorithm for predicting future success based on past and current behavior. In the various embodiments that utilize dynamic periodic goal setting, various approaches may be used to provide such dynamic behavior. For example, in some embodiments, a machine learning approach (e.g., regression, neural networks, Bayesian networks, etc.) may be used to learn appropriate periodic goals (or other sub goals) for a given end-goal and, in some embodiments, wearer.

FIG. 5 illustrates an example of hardware 500 for implementing a locking wearable device such as the user wearable device 420. As shown, the device 500 includes a processor 505, ache and system memory 515, user interface 520, communication interface 525, one or more sensors 530, a lock actuator 535, and a storage 540 interconnected via one or more system buses 505. It will be understood that FIG. 5 constitutes, in some respects, an abstraction and that the actual organization of the components of the device 500 may be more complex than illustrated.

The processor 510 may be any hardware device capable of executing instructions stored in cache/system memory 515 or storage 540 or otherwise processing data. As such, the processor may include a microprocessor, field programmable gate array (FPGA), application-specific integrated circuit (ASIC), or other similar devices. In some embodiments, such as those relying on one or more ASICs, the functionality described as being provided in part via software may instead be hardwired into the operation of the ASICs and, as such, the associated software may be omitted.

The cache/system memory 15 may include various memories such as, for example L1, L2, or L3 cache or system memory. As such, the memory 515 may include static random access memory (SRAM), dynamic RAM (DRAM), flash memory, read only memory (ROM), or other similar memory devices.

The user interface 520 may include one or more devices for enabling communication with a user. For example, the user interface 520 may include a display, buttons, touchscreen, a speaker, a vibrator, a microphone, a camera, haptic engine, etc. Some such user interface 520 devices may double as sensors 530; for example, the microphone or camera may also be used to gather physiological data for use by the parameter extraction engine. The sensors 530 may include various other devices such as, for example, motion sensors (accelerometers, gyroscopes, etc.), temperature sensors (e.g., a thermistor), conductance sensors (e.g. for measuring galvanic skin resistance), or any other sensor hardware for obtaining data related to physiological parameters including those described herein.

The communication interface 525 may include one or more devices for enabling communication with other hardware devices. For example, the network interface 525 may include a network interface card (NIC) configured to communicate according to the WiFi or Ethernet protocols. Additionally, the communication interface 525 may implement a TCP/IP stack for communication according to the TCP/IP protocols. In some embodiments, the communication interface 525 may include an NFC, Bluetooth, or other short range wireless interface. Various alternative or additional hardware or configurations for the communication interface 525 ill be apparent.

The lock actuator 535 may be any device that is capable of selectively holding the clasp in a locked or unlocked state. The lock actuator 535 may include an interface to the system bus that receive, for example, a simple signal such as a pulse-width-modulation (PWM) signal to enable the processor to control the operation thereof. The lock actuator may be, for example, one or more solenoids, one or more electromagnets, or other devices capable of mechanical movement in response to electronic control.

The storage 540 may include one or more machine-readable storage media such as read-only memory (ROM), random-access memory (RAM), magnetic disk storage media, optical storage media, flash-memory devices, or similar storage media. In various embodiments, the storage 540 may store instructions for execution by the processor 510 or data upon with the processor 510 may operate. For example, the storage 540 may store an operating system 261 for controlling various basic operations of the hardware 500. Sensor data reporting instructions 542 may be used by the processor to poll the sensors 530 for data, store the data in a sensor data record 546, and, in some embodiments, subsequently transmit the sensor data to another device such as a wearable device management framework or parameter extraction engine. Locking instructions 543 may be used by the processor 510 to interpret instructions to lock or unlock the device and provide corresponding signals to the lock actuator 535. The locking instructions 544 also include early unlocking instructions 544 which may additionally use previously receive authorization tokens to ensure that the instructing device is authorized and sufficient to direct unlocking of the device. Notification instructions 545 may be used by the processor 510 to interpret received communication instructions and control the user interface to output any communications specified thereby.

It will be apparent that various information described as stored in the storage 260 may be additionally or alternatively stored in the cache/system memory 230. In this respect, as will be understood, the cache/system memory 515 and storage 540 all qualify as memory devices. Further, the cache/system memory 230 and storage 260 may both be considered to be “non-transitory machine-readable media.” As used herein, the term “non-transitory” will be understood to exclude transitory signals but to include all forms of storage, including both volatile and non-volatile memories.

While the hardware device 500 is shown as including one of each described component, the various components may be duplicated in various embodiments. For example, the processor 510 may include multiple microprocessors that are configured to independently execute the methods described herein or are configured to perform steps or subroutines of the methods described herein such that the multiple processors cooperate to achieve the functionality described herein.

FIG. 6 illustrates a perspective view of an example of a locking wearable device 600. The locking wearable device 600 may correspond to the user wearable device 420 or the hardware device 500. As shown, the locking wearable device 600 is of a watch or bracelet form factor. The locking wearable device 600 includes a central hub 610 for housing various hardware components such as, for example, a processor, memories, sensors, output devices, etc. The central hub 610 is attached to two band portions 620, 630 which include female and male mating ends 623, 633, respectively. A slot 623 on one band 620 includes therein a clasp and lock actuator 626 that is electronically controllable by the components of the hub 610 via an electronic connection 629 (e.g., a wire that may carry a PWM signal). A projection 633 is sized to be received within the slot 623 and includes a recess 636 for receiving the clasp and thereby resisting disengagement with the slot 623. Thus, as shown, the locking wearable device 600 may be wrapped around the wearer's wrist and the mating ends 623, 633 engaged to attach the locking wearable device 600 to the wearer. The clasp and locking actuator 626 may prevent or otherwise resist disengagement until the components of the central hub 610 instruct the clasp and locking actuator 626 to allow such disengagement.

FIG. 7A illustrates a cross-section view of a locking wearable device 700 showing a first example of a clasp and locking actuator. Specifically, the cross section may be taken across the female end of a band 705, looking into the slot 710. A lower recess 720 and upper recess 725 are provided for housing, at least in some configurations, some or all of a clasp 723 and locking actuator 727. As shown, the clasp 723 and locking actuator 727 are tubular and arranged in a telescoping configuration. As such, the locking actuator 727 provides a portion of a channel in which the clasp 723 travels. The upper recess 715 is sized and positioned to receive the clasp 723 when fully extended. Thus, when the clasp is fully extended through a recess of the male end (not shown), the clasp is received in both the upper recess 715 and the locking actuator 727 and thereby supported at both ends against removal from the slot if the male end (not shown) were attempted to be forcefully pulled out.

Various hardware embodiments for the clasp 723 and locking actuator 727 will be apparent. For example, the two components 723, 727 together may form a solenoid and, when current is passed through the locking actuator 727, a magnetic field is produced to repel the clasp 723 upward as long as the current continues to flow. Alternatively, an electro magnet may be provided at the bottom of the actuator tube 727 to effect a similar repulsion. Thus, in a non-powered state, the clasp 723 is free to retract into the channel provided by the locking actuator 727 (e.g., under force of gravity) and, as such, the wearable device may be generally unlocked when unpowered or whenever the processor does not assert a signal to the lock actuator 727. Alternatively, the lock actuator 727 may include or otherwise receive signals from a controller that allows the processor to toggle the lock state, such that the processor need not continuously assert the locking signal. It will be apparent that the operation may be reversed such that the default state of the clasp is locked when unpowered by, for example, using a spring to bias the clasp 723 upward and orienting the solenoid to force the clasp downward when current is applied, placing an attractive electromagnet at the bottom of the lower recess 720, or placing a repulsive electromagnet in the upper recess. Various additional variations will be apparent.

FIG. 7B illustrates a cross-section view of a locking wearable device 730 showing a second example of a clasp and locking actuator. While FIG. 7A illustrates various examples where the locking actuator allows movement of the clasp to the open position by actively moving the clasp (either by application or removal of forces), FIG. 7B illustrates various examples where the locking actuator allows movement of the clasp to the open position by removing impediments to manual movement of the clasp to the open position.

The cross section may be taken across the female end of a band 735, orthogonal to the direction of the cross-section of FIG. 7A, looking across the slot 740. As shown, a locking actuator 745 is provided in the upper wall of the slot 740 and a clasp 750 is provided within the slot 740. The clasp 750 includes a catch 752, a nose 754, and a pivot point 756. The clasp 750 is shown in a transition between open and closed positions. When in an open position, the catch 752 lays substantially parallel with the floor of the slot 740 while the nose 754 is perpendicular therewith. As the male end (not shown) is inserted into the slot 740, it abuts and pushes the nose 754 forward, causing the clasp 750 to rotate around the pivot point 756 and the catch to move upward into an aperture of the male end. As the catch 752 passes the lock actuator 745, the telescoping lock actuator 745 moves (e.g., under force of the catch 752) to allow the catch to pass 752 and then returns to its original, extended position. Thereafter, if the male end is pulled outward, the catch 752 abuts the extended portion of the lock actuator 745 which, in this opposite direction, does not give way and keeps the clasp 750 in the closed position. If the processor signals the lock actuator 745 to unlock, it may retract the extended portion (e.g., via operation of a solenoid or attractive electromagnet). As such, operation of the lock actuator itself does not move the clasp 750 into the open position, but removes the impediment to the wearer pulling the male end to thereby moving the clasp 750 into the open position. Various alternative hardware for accomplishing similar functionality (e.g., a ratchet wheel) will be apparent. Additionally, various other clasp and locking actuator mechanisms will be apparent; various embodiments are capable of operating in conjunction with any electronically controllable lock for a wearable device.

FIG. 8 illustrates an example of a method 800 performed by a sensor device for reporting sensor data. It will be understood that, while various examples of methods and flowcharts are detailed herein, that various modifications to these methods and flowcharts may be made while still performing the same or similar functionality. For example, in some embodiments, the steps illustrated may be reordered or performed in parallel with each other.

The method 800 may be performed by a sensor device 150, may correspond to the sensor data reporting instructions 542 of FIG. 5, and may be performed periodically such as, for example, upon expiration of a timer, reaching a time scheduled for the task, or request by another device.

The method 800 begins in step 806 and proceeds to step 810 where the sensor device polls any sensors that it carries or otherwise manages (e.g., external sensors) for new sensor data. Then, in step 815, stores the new sensor data, for example, in a new timestamped record. Then, in step 820, the sensor device determines whether sensor data should be reported to another device. For example, where the sensor device is configured to report new data on a periodic basis that is longer than the period at which the method 800 runs, the sensor device may determine whether the longer period has expired. As another example, the sensor device may decide that data is to be reported whenever the number of “fresh” (i.e., not-yet-reported) sensor data records exceeds a predetermined number. As yet another example, the sensor may perform some analytics to determine whether the acquired data is sufficiently “interesting” to be reported (e.g., hold accelerometer data until it exceeds a minimum threshold). If the sensor data is to be reported, the method 800 proceeds to step 825 where the sensor device transmits and fresh sensor data records to one or more remote devices and marks those records as no longer fresh so that they will not be transmitted the next time reporting occurs. The method then proceeds to end in step 830.

In some embodiments, the sensor device may immediately report all new sensor data rather than saving multiple records to report. In such embodiments, one or more of steps 815 and 820 may be omitted. For example, the sensor device may proceed directly from step 810 to transmit the new data in step 825.

In some embodiments, the sensor device may not report sensor data to other devices at all. For example, in some embodiments, the parameter extraction engine 160 or habit rules engine 120 may be collocated on the same hardware as the sensor device. In such embodiments, steps 820 and 825 may be omitted and the local parameter extraction engine 160 or habit rules engine 120 may simply use the records created among the various executions of step 815.

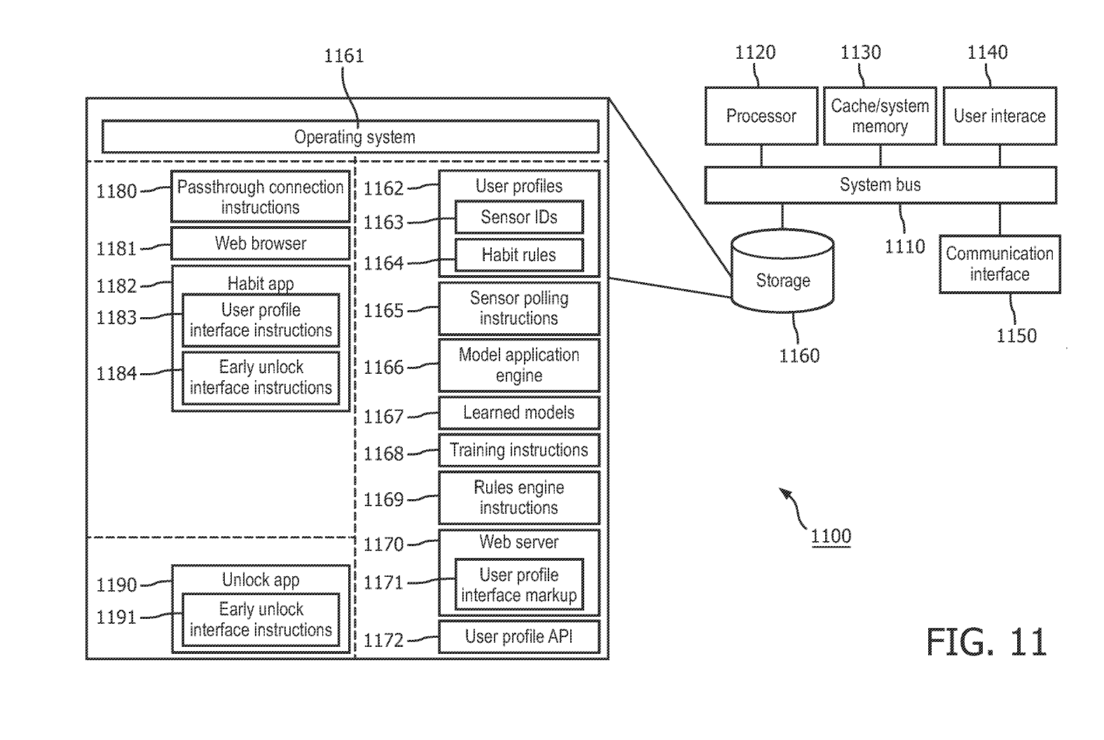

FIG. 9 illustrates an example of a method 900 performed by a locking wearable device for handling instructions from a habit rules engine. Specifically, the method 900 may be performed by a device that combines a locking wearable device 130 and output device 140 in the form of a vibrator. Various modifications to the method 900 for other hardware arrangements will be apparent. The method 900 may correspond to the locking instructions 543 and notification instructions 545. The method 900 may be performed, for example, in response to receiving an instruction from a remote device.

The method 900 begins in step 905 and proceeds to step 907 where the device determines whether the instruction is from an authorized device. For example, the device may determine whether the instruction was received from a known habit rules engine by, for example, verifying a digital signature, password, or other token included with the instruction message. If the instruction is not authorized, the device may simply ignore it and the method 900 may proceed to end in step 950.

If, on the other hand, the instruction is sufficiently authorized, the method 900 proceeds to step 910 where the device determines whether the instruction message carries a locking indication that indicates that the device should be unlocked. If so, the device, in step 915, controls the lock actuator to unlock the device. For example, depending on the implementation and configuration of the lock actuator and clasp, the device may assert a continuous signal to hold the lock actuator in an unlocked state, cease asserting a signal to revert the lock actuator to a by-default unlocked state, assert a discrete signal to toggle the lock actuator into a continuing unlocked state, or perform no action (or change to an ongoing action) if the lock actuator is currently in an unlocked state.

If the instruction message does not carry a locking indication regarding unlocking the device, the method proceeds from step 910 to step 920 where the device determines whether the instruction message carries a locking indication that indicates that the device should be locked. If so, the device, in step 925, controls the lock actuator to lock the device. For example, depending on the implementation and configuration of the lock actuator and clasp, the device may assert a continuous signal to hold the lock actuator in a locked state, cease asserting a signal to revert the lock actuator to a by-default locked state, assert a discrete signal to toggle the lock actuator into a continuing locked state, or perform no action (or change to an ongoing action) if the lock actuator is currently in an locked state.

In step 930, the device determines whether the instruction message includes a communication indication indicating that a vibration should be output to the user. If so, the device reads vibration characteristics from the received message such as, for example, number of vibrations in a group, length of vibrations, interval between vibrations in the group, or period to elapse between repeating the group of vibrations. Then, in step 945, the device controls the local vibrator to vibrate according to the vibration characteristics by, for example, immediately controlling the vibrator or scheduling a repeating vibration task. The method then proceeds to end in step 950.

According to various embodiments, the device executing the method may include output devices in alternative or addition to a vibrator. Various modifications to the method to enable receiving and handling communication indications for the additional or alternative communication types enabled by these other devices will be apparent. For example, steps for reading a text message from the communication indication and outputting the text message via a display device, when available, may be included after step 945 or in place of steps 930-945.

In some embodiments, the locking wearable device 130 or output device 140 may be collocated on the same hardware as the habit rules engine 120 and, as such, may not receive an instruction message from an external device. Various modifications for embodiments whether locking indications or communication indications are received through operation of a local habit rules engine will be apparent.

In some embodiments, locking indications and communication indications may be handled by separate methods, where the output device and locking wearable device are implemented on separate hardware or even in some embodiments where the output device and locking wearable device are implemented on the same hardware. Logical points for separation of the steps will be apparent. For example, steps 930-945 may be removed from method 900 and implemented as a separate method that corresponds only to the notification instructions 545, while the remaining steps of the method 900 may correspond only to the locking instructions 543.

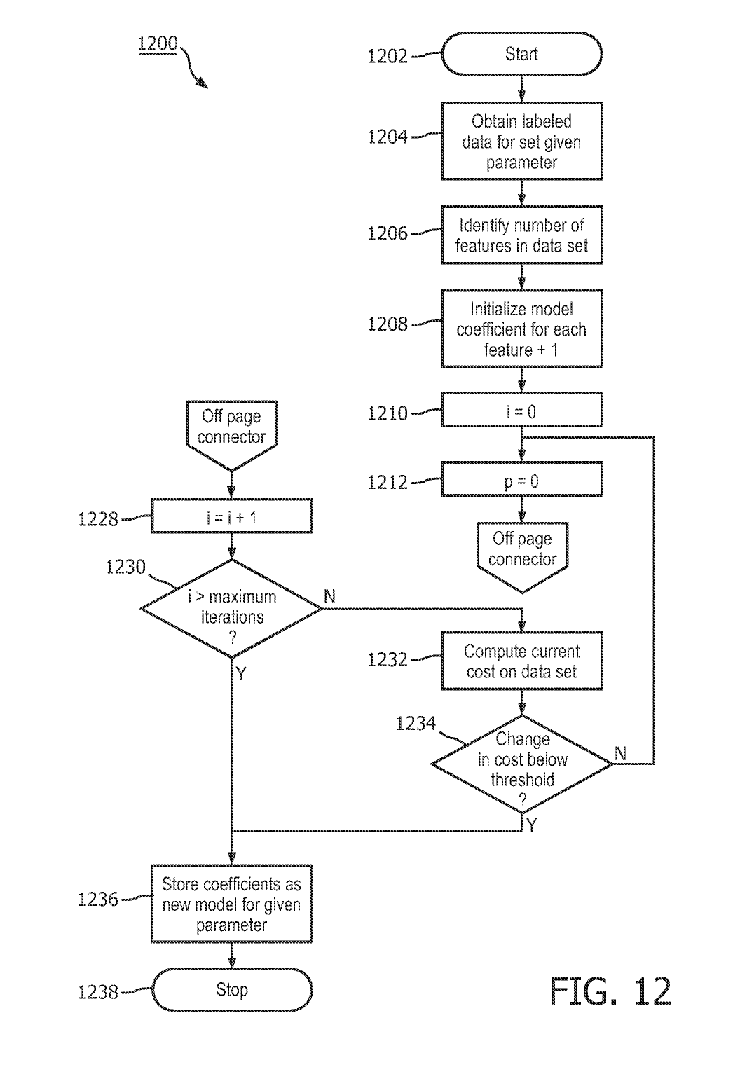

FIG. 10 illustrates an example of a method 1000 performed by a locking wearable device for handling early unlock instructions. This method 1000 may correspond to the early unlock instructions 544 and may be performed in response to receiving an early unlock instruction from an early unlock authorization device. According to this method 1000, the locking wearable device has previously received one or more authorization tokens 547 all of which are needed (according to this illustrated embodiment) to effect early unlock of the device.

The method 1000 begins in step 1005 and proceeds to step 1010 where the locking wearable device extracts an authorization token (e.g., an identifier assigned to the early unlock authorization device by the habit rules engine, a digital signature of the early unlock authorization device, a password, etc.) from the received early unlock instruction. Next, in step 1015, the locking wearable device determines whether the received token matches any of the stored authorization tokens (thereby indicating that the early unlock instruction was received from an early unlock device that is actually at least partially authorized to send the instructions). It will be apparent that different forms of “matching” may be performed in step 1015 such as, for example, determining whether two tokens are equivalent or decrypting a received digital signature authorization token and comparing the result to a stored token. If there is a match, the stored token is marked as having been received.

Next, the locking wearable device begins to determine whether the device should be unlocked by, in step 1025, determining whether a sufficient number of tokens have been marked as received. For example, the locking wearable device may determine that when predefined number or proportion of stored tokens has been received, that a sufficient number has been received. In other embodiments, the locking wearable device may determine that a sufficient number of tokens have been received only when all stored tokens are marked. In some any one of multiple stored tokens may be sufficient to unlock the device; in some such embodiments, steps 1020 and 1025 may be omitted and the method may proceed from step 1015, via the positive branch, directly to step 1030.

In step 1030, the locking wearable device controls the lock actuator to unlock the device. For example, depending on the implementation and configuration of the lock actuator and clasp, the device may assert a continuous signal to hold the lock actuator in an unlocked state, cease asserting a signal to revert the lock actuator to a by-default unlocked state, assert a discrete signal to toggle the lock actuator into a continuing unlocked state, or perform no action (or change to an ongoing action) if the lock actuator is currently in an unlocked state. The locking wearable device then unmarks all stored tokens in step 1035 and the method 1000 proceeds to end in step 1040.

In some embodiments, only a single device may be authorized to send the early unlock instruction and, as such, mere receipt of the instruction is sufficient to unlock the locking wearable device. In some such embodiments steps 1020, 1025, and 1035 may be omitted. In some embodiments, the locking wearable device may not perform any verification and, as such, steps 1010, 1015, 1020, 1025, and 1035 may be omitted. Further, in some embodiments, the early unlock instruction may not be received directly from an early unlock authorization device and, instead, may be received from another device such as the habit rules engine. In such embodiments, the other device may receive instructions from the early unlock authorization device(s), perform a method similar to method 1000, and transmit unlocking instructions to the locking wearable device.

In various alternative embodiments, locking wearable device may be unlocked early without authorization from the supporter. For example, the wearer may wish to temporarily pause their training program based on, e.g., illness. In some such embodiments, the wearer may indicate a desire to put the program on hold for a time and the locking wearable device 130 will unlock in response, thereby enabling removal. After the passage of a preset time or time indicated by the user, a reminder will be provided to the user to put the locking wearable device back on to resume the program. For example, the locking wearable device may begin to emit sounds, vibrations, visual effects, or other reminders. In some embodiments, the reminder emitted from the locking wearable device may be chosen by the wearer or may be selected to be annoying to the wearer. In some embodiments, the wearer's smart phone or other device may provide the reminder via a habit training app such as a message or the same (in some embodiments, annoying) reminders described with respect to the locking wearable device. In some embodiments, the reminder may render the wearer's device (e.g., smart phone) useless or usable with limited functionality until the wearer relocks that wearable device or indicates that the pause to the program should be extended. In some embodiments, the locking wearable device may be adapted to determine whether it is locked yet not currently worn by the user or to prevent locking unless worn by the user (e.g., if the user locks the band without first placing it around their wrist). For example, the locking wearable device may determine whether one or more physiological parameters normally gathered by the device (e.g., heart rate or motion indicating that the device is being worn and not placed on a table or being moved while held in the wearer's hand) are available and, if not, prevent locking of the device or prevent cessation of reminders due to a paused program upon locking.

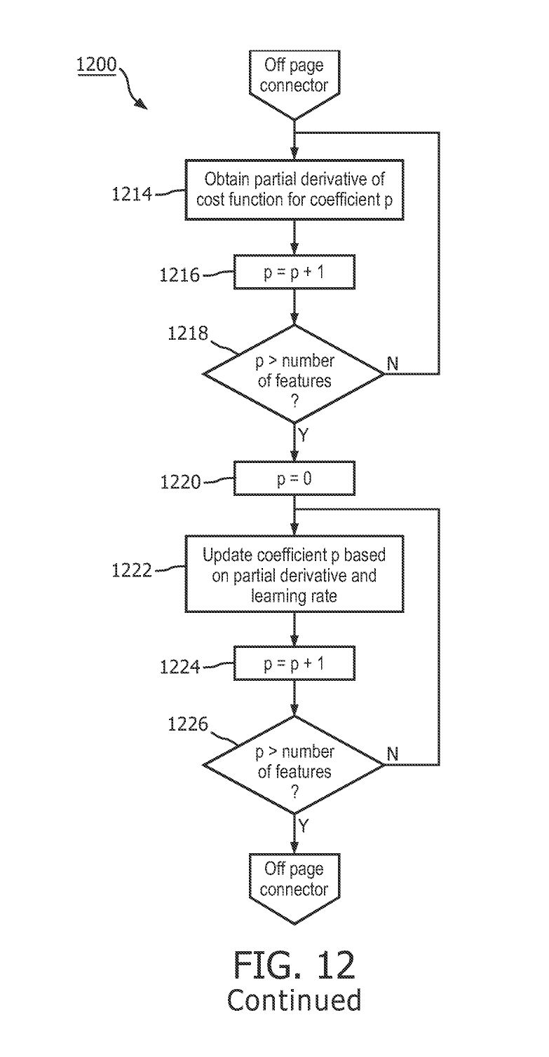

FIG. 11 illustrates an example of hardware 1100 or implementing various devices that participate in the various systems described herein. For example, the hardware 1100 may implement a habit training VM 440, a user mobile device 430, or a supporter mobile device 450. As shown, the device 1100 includes a processor 1120, cache/system memory 1130, user interface 1140, network interface 1150, and storage 1160 interconnected via one or more system buses 1110. It will be understood that FIG. 11 constitutes, in some respects, an abstraction and that the actual organization of the components of the device 1100 may be more complex than illustrated.

The processor 1100 may be any hardware device capable of executing instructions stored in memory 1130 or storage 1160 or otherwise processing data. As such, the processor may include a microprocessor, field programmable gate array (FPGA), application-specific integrated circuit (ASIC), or other similar devices. In some embodiments, such as those relying on one or more ASICs, the functionality described as being provided in part via software may instead be hardwired into the operation of the ASICs and, as such, the associated software may be omitted.

The cache/system memory 1130 may include various memories such as, for example L1, L2, or L3 cache or system memory. As such, the memory 1130 may include static random access memory (SRAM), dynamic RAM (DRAM), flash memory, read only memory (ROM), or other similar memory devices.

The user interface 1140 may include one or more devices for enabling communication with a user such as an administrator. For example, the user interface 1140 may include a display, a mouse, a keyboard, a touchscreen, buttons, camera, microphone, vibrator, haptic engine, etc. In some embodiments, the user interface 1140 may include a command line interface or graphical user interface that may be presented to a remote terminal via the communication interface 1150.

The communication interface 1150 may include one or more devices for enabling communication with other hardware devices. For example, the network interface 1150 may include a network interface card (NIC) configured to communicate according to the Ethernet protocol. Additionally, the communication interface 1150 may implement a TCP/IP stack for communication according to the TCP/IP protocols. Various alternative or additional hardware or configurations for the communication interface 1150 will be apparent. In some embodiments, the communication interface 1150 may include an NFC, Bluetooth, or other short range wireless interface. Various alternative or additional hardware or configurations for the communication interface 1150 will be apparent.

The storage 1160 may include one or more machine-readable storage media such as read-only memory (ROM), random-access memory (RAM), magnetic disk storage media, optical storage media, flash-memory devices, or similar storage media. In various embodiments, the storage 1160 may store instructions for execution by the processor 1120 or data upon with the processor 1120 may operate. For example, the storage 1160 may store an operating system 1161 for controlling various basic operations of the hardware 1100.

Where the hardware 1100 implements a parameter extraction engine 110 and habit rules engine 120 (e.g., the habit training VM 440), the storage 1160 may also store one or more user profiles 1162 identifying the wearers or locking wearable devices that currently participate in the habit training system. The user profiles 1162 include one or more sensor identifiers 1163 for identifying sensor devices or individual sensors (e.g., integrated into the locking wearable device or as standalone devices) associated with each user and one or more habit rules 1164 for defining when the locking wearable device is to be unlocked on when communications should be output to via an output device (e.g., the locking wearable device or another device). Sensor polling instructions 1165 may be used by the processor to periodically obtain sensor data for use in parameter extraction. For example, the sensor polling instructions 1165 may request, from a wearable device management framework, sensor data associated with each of the sensor IDs 1163 for a particular user. After obtaining sensor data, a model application engine 1166 may use the sensor data in conjunction with appropriate learned models 1167 (e.g., by plugging appropriate sensor values into parameter-specific learned mathematical functions that serve as learned models 1167).

Training instructions 1168 enable the processor to initially create and, in some embodiments, further adapt the learned models 1167. In some embodiments, the training instructions 1168 may be executed by another device to create the models, which are then loaded into the storage 1160 for use by the model application engine 1166. In some embodiments, generalized learned models 1167 may be adapted to specific users based on feedback to create user-specific learned models which may be stored as part of the user profile 1162. For example, a user may train the parameter extraction engine to use audio sensor data to identify when that user is practicing playing music to generate a new mode 1167 or may train existing models to be more accurate to that specific user.

Rules engine instructions 1169 may periodically evaluate habit rules 1164 against sensor data retrieved from by the sensor polling instructions 1165 or extracted by the model application engine 1166 to determine when locking or communication indications should be provided to a locking wearable device or output device. In some embodiments, a web server 1170 and associated user profile interface markup 1171 may provide a browser-based interface for a habit setting device to define the habit rules 1164 or other aspects of the user profile 1162. Additionally or alternatively, a user profile API 1172 may communicate with a habit app or other program or website for a habit setting device to perform the same or similar actions.

Where the hardware 1100 implements a habit setting device 110 (e.g. the user mobile device 430), the storage 1160 may include passthrough connection instructions in some embodiments to facilitate communication between a locking wearable device, other sensor devices, and other devices on the network. In some embodiments, the passthrough connection instructions 1180 may be a configuration of the operating system 1161 to provide such functionality, while in some embodiments the passthrough connection instructions may be part of the habit app 1182. A web browser 1181 may also enable communication with the web server 1170 to accomplish the functionality described above.

A habit app 1182 may include user profile interface instructions 1183 for communicating via the user profile API 1172 to create, modify, or delete the user profile 1162 such as, for example, linking sensor IDs 1163 and defining habit rules 1164. Early unlock interface instructions 1184 may provide an interface and related backend instructions for either sending an early unlock indication to a locking wearable device (e.g., upon pressing an early unlock button by the user) or for sending a request to an early unlock authorization device to send such an early unlock indication to the locking wearable device.

Where the hardware implements an early unlock authorization device, the storage 1160 stores an unlock app 1190 which, in some embodiments, may be the same as the habit app 1181. In other words, in some embodiments a user may use the same app to manage their own user profile and to approve or deny early unlock requests for other users. The unlock app 1190 includes early unlock interface instructions 1191 for indicating when another user has requested an early unlock and then sending a locking indication or communication indication based on the user's input.

Various modifications to these arrangements will be apparent. For example, in some embodiments rather than using an app for early unlock functionality, the web server 1170 may provide markup for defining an early unlock interface which may be accessed by a web browser 1181 or a web browser of the early unlock authorization device (not shown), to perform similar functionality to that described herein with respect to the early unlock interface instructions 1184, 1191.

It will be apparent that various information described as stored in the storage 1160 may be additionally or alternatively stored in the memory 1130. In this respect, the memory 1130 may also be considered to constitute a “storage device” and the storage 1160 may be considered a “memory.” Various other arrangements will be apparent. Further, the memory 1130 and storage 1160 may both be considered to be “non-transitory machine-readable media.” As used herein, the term “non-transitory” will be understood to exclude transitory signals but to include all forms of storage, including both volatile and non-volatile memories.

While the host device 1100 is shown as including one of each described component, the various components may be duplicated in various embodiments. For example, the processor 1120 may include multiple microprocessors that are configured to independently execute the methods described herein or are configured to perform steps or subroutines of the methods described herein such that the multiple processors cooperate to achieve the functionality described herein. Further, where the device 1100 is implemented in a cloud computing system, the various hardware components may belong to separate physical systems. For example, the processor 1120 may include a first processor in a first server and a second processor in a second server.

FIG. 12 illustrates an example of a method 1200 performed by a parameter identification engine or a separate model creation device for creating a parameter model. In particular, the method 1200 may correspond to the training instructions 1168 and may generally describe a liner regression or logistic regression approach to model training for parameter extraction. Various alternative approaches to model training or parameter extraction will be apparent such as, for example, programmer-defined algorithms, neural networks, Bayesian networks, etc.

The method begins in step 1202 and proceeds to step 1204 where the device obtains a labeled data set for a given parameter for which a model is to be created. Various approaches for training a model from an unlabeled training set will be apparent. In various embodiments, the training set may include a number of records of training examples that specify one or more features and the appropriate parameter to be extracted for that feature set. According to various embodiments, features may include sensor data or other parameters extracted according to other models. For example, a training set may include two features, an average heart rate and accumulated accelerometer motion, associated with an energy expenditure value measured or estimated to correspond to each set of values. In various embodiments, the training set may be created from real-world data gathering activities such as gathering sensor data and measuring the parameter at the same time the sensor data was gathered according to another method to create a training example record.

In step 1206, the device identifies the number of features identified in the data set and, in step 1208, initializes a set of coefficients to be used in the resulting model. According to various embodiments, a coefficient is created for each feature along with one additional coefficient to serve as a constant. Where the model is being trained to output a numerical value, a linear regression approach may be utilized, wherein the final model function may take the form of

h(X)=θ₀+θ₁x₁+θ₂x₂ . . .

where X is the set of features {x₁, x₂, . . . } and the coefficients {θ₀, θ₁, θ₂, . . . } are to be tuned by the method 1200 to provide as output an appropriate value estimation of the parameter, consistent with the trends learned from the training data set. Where the model is being trained to output an indication of whether or not a condition is present (e.g., user is running, cycling, etc.), the final model function may incorporate the Sigmoid function as follows:

$$h ( X ) = \frac{1}{( 1 + {e}^{- h ( {θ}_{0} + {θ}_{1} {x}_{1} + {θ}_{2} {x}_{2} … )} )}$$

where tuning of the coefficients results in the function h(X) outputting a value between 0 and 1 that serves as an estimation of the probability of the parameter to which the model is trained being true or present. According to various embodiments, the coefficients are all initialized to values of zero. It will be apparent that in some embodiments, additional features for inclusion in h(X) (and associated coefficients) may be constructed from the features in the training set such as, for example, x₁² or x₁x₂.

The method begins to train the coefficients by initializing two loop variables, i and p, to 0 in steps 1210, 1212 respectively. Then, in step 1214, the device obtains a partial derivative of the cost function, J(θ), on the current coefficient, θₚ, where the cost function may be defined in some embodiments as

$$J ( θ ) = \frac{1}{2} ( {\sum }_{j = 1}^{m} {( {h}_{θ} ( {x}^{( j )} ) - {y}^{( i )} )}^{2}$$

where m is the number of training examples in the training data set, h_{θ}(x) is the trained function using the current coefficient set θ, x^{(j)} is the set of features for the j^{th} training example, and y^{(j)} is the desired output (i.e., the label) for the j^{th} training example. Thus, following a batch gradient descent approach, the partial derivative on coefficient p (θₚ) may be

$${\sum }_{j = 1}^{m} ( {y}^{( i )} - {h}_{θ} ( {x}^{( j )} ) ) {x}_{p}^{( j )}$$

where xₚ^{(j)} is the p^{th} feature in the j^{th} training example (or when p=0, xₚ^{(j)}=1).

In step 1216, the device increments p and, in step 1218, the device determines whether all coefficients have been addressed in the current loop by determining whether p now exceeds the total number of features to be included in h(X). If not, the method loops back around to step 1214 to find the next partial derivative term.

After all partial derivatives are found for the current iteration, the method proceeds to reset the loop variable p to zero in step 1220. Then, in step 1222, the device updates the p^{th} coefficient, θₚ, based on the corresponding partial derivative found in step 1214 and based on a preset learning rate. For example, the device may apply the following update rule:

$${θ}_{p} = {θ}_{p} + α * {\sum }_{j = 1}^{m} ( {y}^{( i )} - {h}_{θ} ( {x}^{( j )} ) ) {x}_{p}^{( j )}$$

where α is a learning rate such as, for example, 0.1, 0.3, 1 or any other value appropriately selected for the desired rate of change on each iteration.

In step 1224, the device increments p and, in step 1226, the device determines whether all coefficients have been addressed in the current loop by determining whether p now exceeds the total number of features to be included in h(X). If not, the method loops back around to step 1222 to update the next coefficient. Note that according to the method 1200, all partial derivatives are found in a first loop prior to actually modifying the coefficients in a second loop so that the partial derivatives are not taken based on the partially updated values. Other embodiments may not implement such a “simultaneous” update of the coefficients.

After all coefficients are updated, the method proceeds to step 1228 where the variable i is incremented. In step 1230, the device determines whether i now exceeds a predefined maximum number of iterations to ensure that the method 1200 does not loop indefinitely. A sufficiently high maximum number of iterations may be chosen such as 1000, 5000, 100000, etc. If the maximum iterations has not been reached, the method proceeds to step 1232 where the device computes the current cost, using the cost function J(θ), based on the training set. In step 1234, the device determines whether the function h(X) has converged to an acceptable solution by determining whether the change in the cost from the last iteration to the present iteration fails to meet a minimum threshold. If the change surpassed the threshold the method loops back to step 1212 to perform another coefficient update loop. If, on the other hand, the maximum iterations is reached or the cost change is below the minimum threshold, the method 1200 proceeds to step 1236, where the device stores the coefficients as part of the new model for extracting the parameter and the method 1200 proceeds to end in step 1238.

It will be apparent that, in addition to following approaches other than regression, other embodiments may utilize different methods for tuning coefficients in a regression approach other than batch gradient descent. For example, some embodiments may use stochastic gradient descent, wherein each coefficient update is performed based on a single training example (thereby removing the summation from the partial derivative), and the method additionally iterates through each such example. In other embodiments, the normal equations for regression may be used to find appropriate coefficients, using a matrix-based, non-iterative approach where the set of coefficients is computed as

θ=(X^{T}X)⁻¹X^{T}y

where X is a matrix of features from all training examples and y is the associated vector of labels.

FIG. 13 illustrates an example of a method 1300 performed by a parameter identification engine or other model training device for updating a parameter model. As noted above, in various embodiments, real data from the wearer may be used to further adapt parameter models to the individual wearer, providing more accurate results. According to the method 1300, the generic training set may be supplemented or replaced with wearer-specific training examples, such that the method 1200 (or other training method) may be run again to create a more accurate parameter model. The method 1300 may correspond to the training instructions 1168 and may be performed periodically or in response to receiving wearer feedback. Feedback may take various forms such as an explicit indication of what the correct parameter was, an indication that a previous parameter extraction (or other action based on the parameter) was correct or incorrect, or lack of explicit feedback indicating implicit (though potentially weak) approval of the parameter or related outcome.

The method 1300 begins in step 1305 and proceeds to step 1310 where the device obtains the features that were previously used to extract a parameter. For example, the parameter extraction engine may keep records or previously extracted parameters and the features used for the extraction. Alternatively, the feedback may include some or all of the relevant features. Next, in step 1315, the device creates a new training example including the obtained features. In various embodiments, the training example may match the schema of the training examples used in method 1200. Next, in step 1320, the device begins to determine a label for the training example by determining whether the feedback indicates that the previous parameter extraction (or resultant action) was correct. If so, the device labels the training example in step 1325 to match the previously extracted parameter. Otherwise, in step 1330, the device labels the training example as the opposite of the previously extracted parameter, particularly when a logistic regression approach is being employed for the parameter where the result of the model is a classification, e.g., of yes or no (i.e., Boolean). In other embodiments, step 1330 may involve obtaining an alternative label by adjusting the previously extracted parameter in a direction indicated by the feedback or using a value explicitly provided by the feedback. Various additional modifications will be apparent.

In step 1335, the device inserts the new training example into the training set for the parameter in question. In step 1340, the device may determine whether old training entries are to be decayed. For example, the device may decay old entries as long as non-user specific entries remain in the set. Alternatively, decaying of old entries may be a configurable option in the system. In other embodiments, decaying of entries may not be implemented and steps 1340 and 1345 may be omitted. If the entries are to be decayed, the device deletes the oldest training example from the training set for the parameter. Where the training examples are not dated, the device may select (e.g., arbitrarily or based on proximity of features to the new training example) a general training example for deletion. In some embodiments, more than one entry may be deleted in step 1345.

In step 1355, the device updates the model using the updated training set by, for example, invoking method 1200. In other embodiments, the device may wait until a predefined time (e.g., non-peak hours for the parameter extraction engine or training device) or until a predefined number of new training examples have been added before executing step 1355. The method 1300 may then proceed to end in step 1360.

FIG. 14 illustrates an example of a method 1400 performed by a habit rules engine for evaluating habit rules and remotely controlling a locking wearable device. The method 1400 may correspond to the rules engine instructions 1169 and may be executed, for example, periodically or upon receiving new parameters (e.g., sensor data or extracted parameters) for a particular wearer.

The method 1400 begins in step 1402 and proceeds to step 1404 where the habit rules engine retrieves a user profile (e.g., user profile 1162) for the wearer currently being evaluated. Based on the user profile, the habit rules engine identified that relevant sensors for the wearer in step 1406. For example, the habit rules engine may extract all available sensor IDs 1163 from the user profile or may extract only those sensor IDs implicated by the habit rules 1164 or otherwise indicated as a relevant subset in the user profile. Next, in step 1408, the habit rules engine obtains any new data available from the identified sensors to supplement previously retrieved data for the sensors as may be already stored in the user profile or elsewhere. For example, the habit rules engine may send a polling request to the sensor devices identified by the sensor IDs or may send a pull request to a wearable device management framework identifying the sensor IDs for which new data is requested. In step 1410, the habit rules engine retrieves any historical sensor data previously received and other parameters previously extracted, for example, from the user profile.

In step 1412, the habit rules engine obtains a habit rule from the user profile for evaluation. As will be explained in greater detail below, a habit rule may include a criteria portion for evaluating applicability of the habit rule and an action portion for defining one or more actions to be taken when the rule is applicable. In step 1414, the habit rules engine determines whether the criteria includes any references to extracted parameters that need to be obtained prior to criteria evaluation. For example, if the rule requires a comparison involving calories expended, this value should first be obtained. In step 1416, the habit rules engine invokes the parameter extraction engine to extract the needed parameter, for example, according to an extraction algorithm or model relating to that parameter. In some embodiments, such as those wherein a particular parameter is extracted based on other extracted parameters (rather than solely on sensor data), the parameter extraction engine may first extract the premise parameters before extracting the referenced parameter. In step 1418, after the parameter extraction engine returns the requested parameter(s), the habit rules engine determines whether additional referenced parameters remain for extraction. If this is not the last extracted parameter, the method 1400 loops back to step 1416 to obtain additional parameters. Otherwise, the method proceeds to step 1420.

In various alternative embodiments, the method 1400 need not or otherwise does not take steps to obtain sensor data or other parameters. For example, in some embodiments, the parameter extraction engine may operate independently of the habit rules engine and may periodically store extracted parameters and new sensor data in a location accessible to the habit rules engine such as, for example, the user profile. As such, in some embodiments, one or more of the steps for obtaining parameters (e.g., steps 1408, 1414, 1416, 1418) may be omitted or modified to simply read the values from the expected location.

The habit rules engine then proceeds to evaluate the current rule by, in step 1420, determining whether the rule criteria is met. For example, the habit rules engine may determine if one, more, or all conditions defined in the rule criteria evaluate to true when populated with referenced parameter values. If the rule criteria is not met, the habit rules engine determines that the current rule is not applicable and the method 1400 proceeds to step 1422 where the habit rules engine determines whether the user profile includes additional rules that are yet to be evaluated. If the current rule is the last rule, the method 1400 proceeds to step 1424 where the habit rules engine stores the new parameters with the historical parameters. The method then proceeds to end in step 1438. If, on the other hand, there are additional rules to be evaluated, the method 1400 proceeds from step 1422 back to step 1412, where the habit rules engine obtains the next rule from the user profile for evaluation.

When the habit rules engine judges a rule to be applicable in step 1420, the method 1400 proceeds to step 1426 where the habit rules engine creates a new instruction message to be sent to the locking wearable device (or other output device). In step 1428, the habit rules engine determines whether the rule action indicates that the locking wearable device should be unlocked. If so, the habit rules engine marks the rule itself (or rule group to which the rule belongs) as unlocked in step 1429. In step 1430, the habit rules engine determines whether any other rules (or groups thereof) remain locked. If there are no other locks remaining, the method proceeds to step 1431 where the habit rules engine adds an instruction to unlock the locking wearable device to the instruction message. If, on the other hand, the rule action does not indicate an unlock or if additional locked rules remain, the method 1400 skips step 1431 and proceeds to step 1432.

In step 1432, the habit rules engine determines whether the action indicates that a vibration communication should be output to the wearer. If so, the habit rules engine adds to the instruction message a communication indication specifying the vibrations to be output to the user. Then, in step 1436, the instruction message is transmitted to the locking wearable device or other output device. In some embodiments, the instruction message may only be transmitted in step 1436 if at least one instruction was added to the message by operation of the previous steps. In some embodiments, a rule action may involve instructions to be sent to multiple devices; in some such embodiments, multiple instruction messages may be created for each relevant locking wearable device or output device which would then be sent as appropriate. Further, as explained above, various embodiments may enable additional or alternative communications to vibrations; modifications to the method 1400 for transmitting instructions for such other communication types will be apparent.

As shown, the method 1400 will only ever locate one applicable rule. However, in some other embodiments, the habit rules engine may locate and apply multiple habit rules in a single execution of the method 1400. In such embodiments, the method may loop, for example, from step 1436 to step 1422.

As described, the method 1400 enables multiple groups of rules to individually operate and lock the locking wearable device. Such an arrangement may be useful, for example, where the wearer seeks to adopt more than one habit change such as, for example, burning 2000 calories a day and practicing music for at least 1 hour a week. In such an arrangement, the wearer may only wish the locking wearable device to unlock after both habit training programs have completed. In other embodiments, the locking wearable device may unlock as soon as any training program is complete such as, for example, in embodiments wherein the locking wearable device is only to be used for adopting one habit change or only one habit change at a time. In such embodiments, steps 1429, 1430 may be omitted, and the method 1400 may proceed from step 1428 along the affirmative branch directly to step 1431.

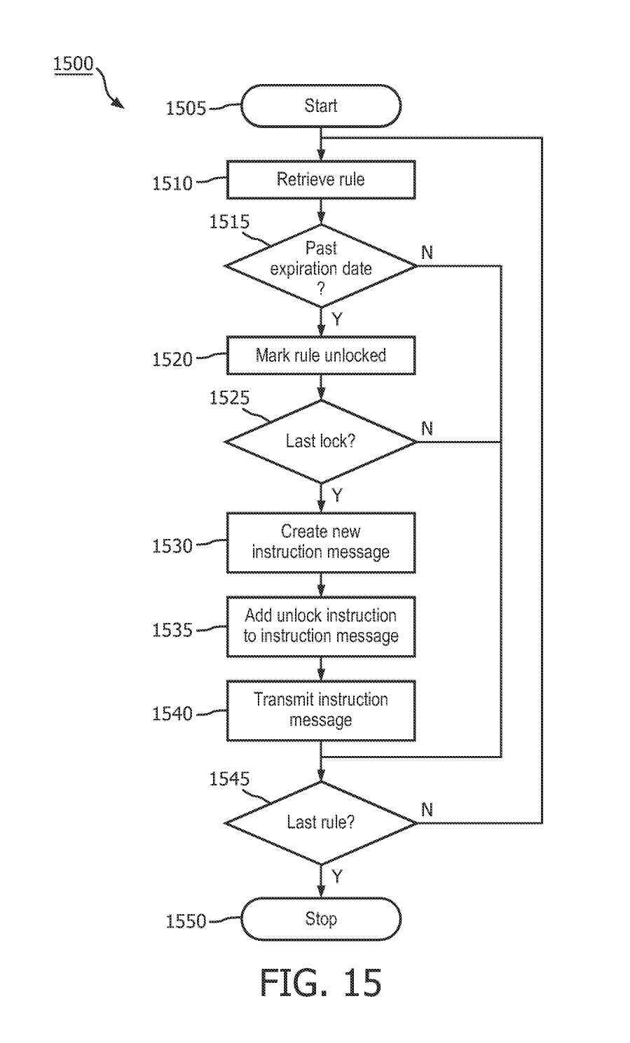

FIG. 15 illustrates an example of a method 1500 performed by a habit rules engine for auditing habit rules for expiration. The method 1500 may, for example, be performed periodically or upon request and may correspond to the rule engine instructions 1169. n some embodiments, the goals of method 1500 may be accomplished by method 1400 (e.g., expired rules may be flagged as applicable in step 1420) and method 1500 may not be implemented as a separate thread.

In step 1510, the habit rules engine retrieves a rule for a wearer to evaluate for expiration. In step 1515, the habit rules engine determines if the current day and time has surpassed the expiration date of the rule. If so, the rule (or rule group to which it belongs) is marked as unlocked in step 1520. In step 1525, the habit rules engine determines whether additional locks remain. If the just-unlocked rule is the last lock, the habit rules engine creates a new instruction message in step 1530, adds an unlock instruction to the instruction message in step 1535, and transmits the instruction message to the locking wearable device associated with the rule in step 1540. After transmitting the instruction message, determining that additional locks remain, or determining that the rule has not expired, the method proceeds to step 1545 where the habit rules engine determines whether this is the last rule to be audited. If not, and additional rules remain, the method 1500 loops back to step 1510 to audit the next rule. Otherwise, the method 1500 proceeds to end in step 1550.

As previously noted, in various embodiments the habit rules engine may be collocated on the same hardware as a locking wearable device of an output device. As such, when the instructions generated according to methods 1400, 1500 are instructions meant for local hardware, they may not be transmitted via a network or as part of an instruction message. Various modifications for enabling local inter-component communication according to these methods 1400, 1500 will be apparent.

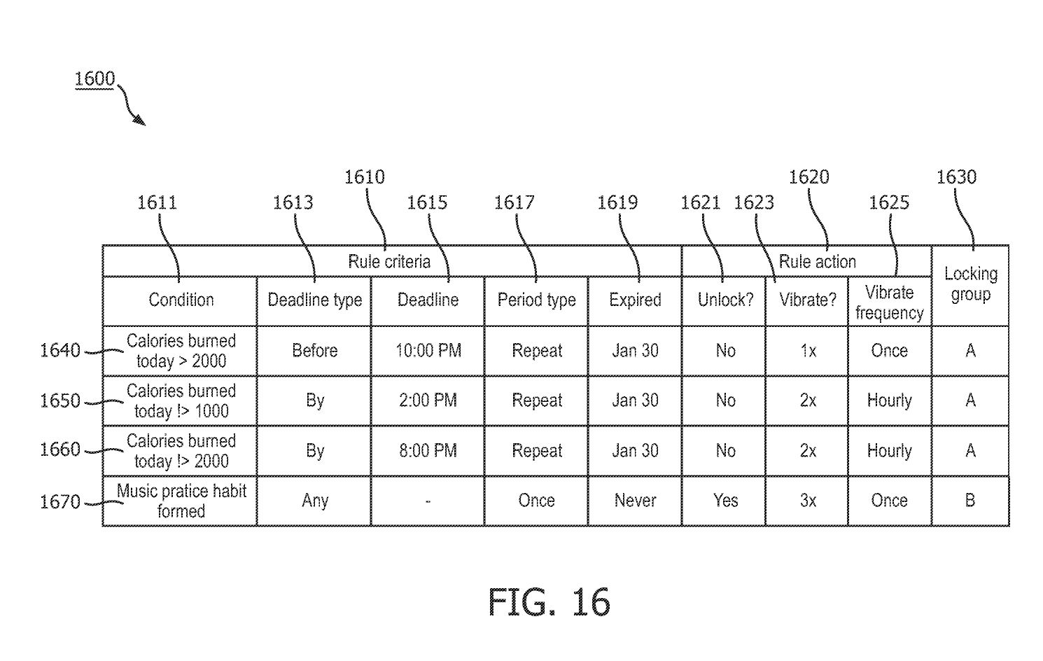

FIG. 16 illustrates an example of a data structure 1600 or storing habit rules. The data structure 1600 may correspond, for example, to habit rules 1164 for a particular user. As shown, the data structure 1600 includes a rule criteria portion 1610 for evaluating rule applicability, a rule action portion 1620 for defining actions to be taken when a rule is applicable, and a rule group field 1630 for grouping rules that lock or unlock the locking wearable device together. As noted above, in some embodiments this grouping functionality is not implemented and the rule group field 1630 may be omitted.

The rule criteria portion 1610 includes a condition field 1611 for defining one or more conditional statements (which may reference parameters) to be evaluated, a deadline type field 1613 and deadline field 1615 for, together, determining what times of day or dates the rule should be evaluated or considered inapplicable (if the condition evaluates affirmatively), a period type field 1617 for defining any repetitive characteristics of the rule, and an expiration field 1619 for defining when the rule (or rule group) expires thus unlocking the locking wearable device.

The rule action portion 1620 includes an unlock field 1621 for specifying whether application of the rule should result in unlocking the locking wearable device, a vibration field 1623 and vibration frequency field 1625 for specifying whether and how a vibration should be output to the user. It will be apparent that additional fields may be defined for further defining the vibration to be output. Further, where rules may result in additional or alternative output types, fields for defining such other communications along with appropriate target output devices will be apparent.

As a first example, three rules 1640, 1650, 1660 belong to a locking group A. A first rule 1640 indicates that every day until January 30, when the user burns over 2000 calories before 10 PM (if at all), the locking wearable device is to vibrate one time to indicate daily goal success. A second rule 1650 indicates that every day at 2 PM, if the user has not burned at more than 1000 calories, that the locking wearable device should vibrate twice every hour (e.g., until the rule 1650 is no longer applicable). Similarly, a third rule 1660 indicates that every day at 8 PM, if the user has not burned at more than 2000 calories, that the locking wearable device should vibrate twice every hour (e.g., until the rule 1650 is no longer applicable). Finally, on January 30, the rules in locking group A will expire and the locking device will be unlocked (provided, in some embodiments, that other locking groups do not remain locked).

As a second example, a second locking group B includes only a single rule 1670 which indicates that, if a parameter extraction model determines generally that the habit of practicing music has been formed (e.g., by monitoring audio data or indications from the wearer that they practiced over successive days and applying a logistic regression model) that the locking wearable device will be unlocked and vibrate three times to indicate habit training success. The rule 1670 does not have an expiration so, if the wearer wishes to remove the wearable device, they must either attain the habit (e.g., as determined by the parameter extraction engine) or request an early unlock from one or more supporters. In some embodiments, rules may be provided without any expiration or unlocking action and, as such, may require an early unlock instruction if the locking wearable device is to be removed.

FIG. 17 illustrates an example of a user interface 1700 for defining habit rules. The user interface 1700 may be presented on a habit setting device 110 and may be generated according to user profile interface instructions 1183 in a habit app 1182 running on the habit setting device 110 or by user profile interface markup 1171 delivered by a web server 1170 to a web browser 1181 running on the habit setting device 110. The user interface 1700 may provide a wearer with an interface by which to define new habit rules or provide sufficient information for the generation of habit rules to be evaluated in connection with the wearer's locking wearable device or output devices.

The user interface 1700 includes a time limit field 1705 for defining an expiration date for the habit rules (or group thereof). The value entered into this field may be copied or used to create a value for, for example, the expiration field 1619. A habit name field 1710 may allow a user to enter or select a user-readable name for a habit change plan. For example, in some embodiments, the habits name field 1710 may be prepopulated with available habit training plans that, when selected, automatically fill in one or more of the remaining controls 1705, 1720-1765 or other aspects of the program. In some embodiments, the wearer may be able to fill out the interface 1700 and save the selections as a new habit training plan for later selection via the habits name field 1710 by the wearer or by other users (e.g., other participants in the habit training systems). A “start and lock” button 1715, when selected, may commit one or more habit rules to the habit rules engine based on the input of the user interface. As previously described, installation of such habit rules may also effect locking of the wearer's locking wearable device, either immediately (e.g., if the wearer is currently wearing the locking wearable device) or upon the next closure of the locking wearable device (e.g., if the locking wearable device is currently not being worn).

A sensors field 1720 may enable the wearer to select one or more sensors that will be utilized by the habit training program. For example, the sensors list 1720 may be prepopulated with all sensors types known or usable by the habits training system or with only those sensors known to be possessed by the wearer. A goals field 1725 may enable the wearer to select, enter, or otherwise define one or more goals (e.g., on time goals, daily goals, weekly goals, etc.). For example, in some embodiments, selection of a sensor in the sensor field 1720 may prepopulate the goals field 1725 with one or more goals related to the selected sensor (or grouping of multiple selected sensors) for selection by the user. Alternatively or additionally, each line 1725 may be selectable by a user to input a custom goal (e.g., by text input or by guided creation via a different interface).

A vibration selector 1730 may enable the wearer to indicate whether the training program will be associated with any vibration communications. Selection of the vibration selection 1730 may enable, reveal, or otherwise provide additional fields 1735-1750. A goal-met vibration field 1735 may receive an indication of whether and how an output device should be vibrated upon meeting one or more of the selected goals in the goal field 1725. For example, as shown, the interface 1700 may indicate that the output device (e.g., the locking wearable device) should be vibrated once when each of the selected goals are met or when all of the goals have been met. In other embodiments, vibration characteristics may be defined for each selected goal individually.

A set of goal-not-met fields 1740, 1745, 1750 may receive an indication of whether and how an output device should be vibrated upon a wearer's failure to meet a particular goal. For example, as shown, the interface 1700 may indicate that that the output device (e.g., the locking wearable device) should be vibrated twice when a goal is not met, and continue to do so every hour until 10 PM until the goal is met. Such definition may be defined for all goals, any goal, or individual goals. Further, it will be apparent that alternative or additional fields for defining other vibration characteristics may be provided in other embodiments. As noted above, various embodiments enable additional or alternative communications to vibration. Modifications to the interface 1700 to enable the wearer to define such other communications and output device targets thereof will be apparent. For example, text fields may be provided for specifying messages or dynamic sources of messages (e.g., a coaching program delivering content cards) to be delivered on meeting of failing to meet a goal.

A supporter mobile device field 1755 may enable the wearer to identify devices of one or more supporters. For example, the field 1755 may receive a user ID of another wearer or other user of the habit training system. As such, the user ID may point to or otherwise be associated with a habit app 1182 or unlock app 1190 to which notifications may be pushed such as, for example, sensor data, extracted parameters, or other information when the “send data” control 1760 is selected or wearer-initiated requests for early unlock when the “early unlock” control is selected 1765. Alternatively, the field 1755 may receive a mobile phone number sufficient to push SMS or other text messages to the supporter device. For example, the text messages may include sensor data, extracted parameters, or other information when the “send data” control 1760 is selected. As another example, a text message may include a link to a web server for providing information or an interface to the supporter such as, for example, an early unlock interface (e.g., if the wearer has requested an early unlock).

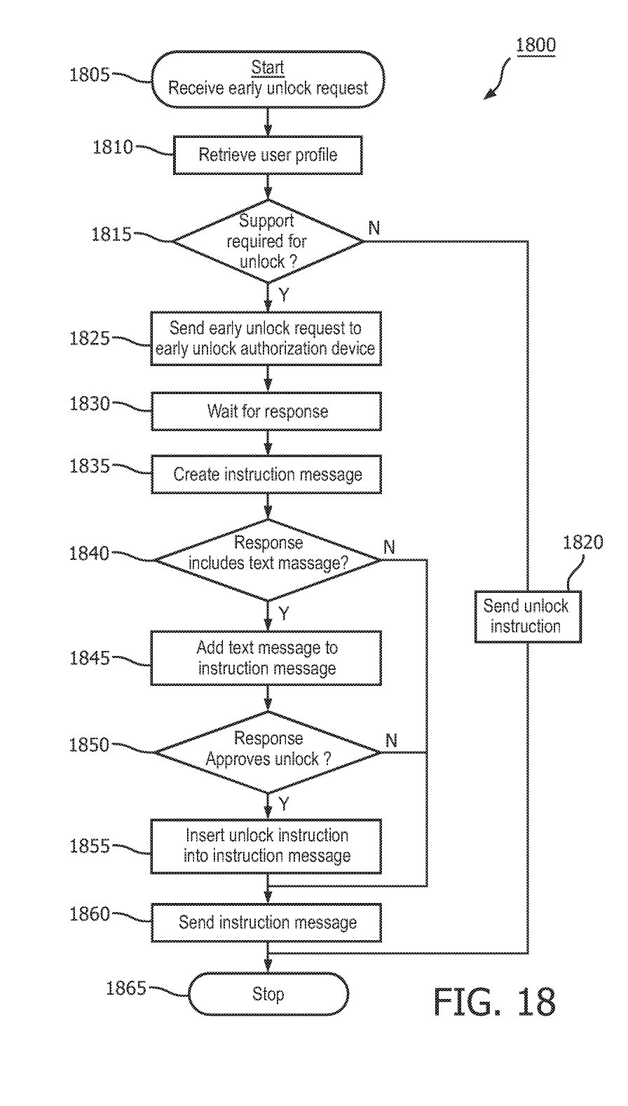

FIG. 18 illustrates an example of a method 1800 performed by a habit rules engine (or other device) for handling an early unlock request. The method 1800 may be performed, for example, in embodiments where the habit rules engine (or other device) acts as an intermediary between the locking wearable device 130 and early unlock authorization device 170 for early unlock operations. In other embodiments, early unlock devices 170 may communicate directly with the locking wearable device (e.g., via the Internet) without any participation by an intermediary and, as such, method 1800 may be omitted.

The method 1805 begins in step 1805 where the device receives a request for early unlock. For example, the request may be received from the locking wearable device or another device of the wearer in response to the wearer indicating (e.g., via a user interface) a desire to unlock the locking wearable device early. Next, in step 1810 the device retrieves a user profile associated with the wearer and, in step 1815, determines whether a supporter is required to unlock the locking wear able device early (e.g., if the wearer initially selected the early unlock control 1765 when initially locking the locking wearable device). If not, the method 1800 proceeds to step 1820 where the device responds with or otherwise transmits an unlock instruction to unlock the locking wearable device. If, on the other hand, approval is required for the early unlock, the device sends the early unlock request to the early unlock authorization device(s) identified by the user profile in step 1825 and waits for a response in step 1830. For example, the device may send an instruction to a habit app or unlock app to display the request with a form for receiving the supporter's input. As another example, the device may send a URL pointing to a web page providing a similar interface.

Once a response approving or denying the early unlock (or the request times out) is received, the method 1800 proceeds to step 1835 where the device creates a new instruction message (or messages) to be delivered to the locking wearable device or other output devices. In step 1840, the device determines whether the response includes a text message (or any other communication) to be output to the wearer and, if so, includes an instruction to output the text message (or other communication indication) in the instruction message in step 1845. In step 1850, the device determines whether the response includes an approval of the early unlock. If so, the device inserts an instruction to unlock the locking wearable device into the instruction message. The device then transmits the instruction message(s) to the appropriate device(s) in step 1860 and the method proceeds to end in step 1865. In various embodiments, the instruction message may include the same types or formats of locking indications or communication indications that may otherwise be generated via application of habit rules; as such, the locking wearable device or output device may apply the same logic (e.g., method 1400) to interpret these indications sent by operation of method 1800 as well.

FIG. 19 illustrates an example of a user interface 1900 or responding to an early unlock request. The user interface 1900 may be displayed, for example, by early unlock instructions 1184,1191 a habit app 1182 or unlock app 1190 executing on an early unlock authorization device. In other embodiments, the user interface 1900 may be displayed by a web browser 1181 rendering markup language delivered by a web server 1170.

As shown, the interface includes a field 1905 identifying the requesting wearer along with approval and denial controls 1910, 1915 for indicating whether the early unlock is to be effected. A message text field 1920 may enable the supporter to enter a text message to be delivered to the wearer upon delivery of the approval or denial. Various additional or alternative fields (not shown) for specifying other communications or target output devices therefor will be apparent. A send button 1925 enables the supporter to commit the input information by sending a message back to, e.g., the locking wearable device, other output devices of the wearer, the habit rules engine, or other device acting as an authorization intermediary.

According to the foregoing, various embodiments provide a system for supporting and reminding users in changing their habits as desired. For example, by providing an electronically lockable/unlockable wearable device, a wearer's commitment to a training program may be enforced and risk of a user giving up early reduced. Further, by providing the wearable device with a vibrator or other output device, the wearer can be actively reminded of the goals they have committed to meet, thereby helping the user to repeatedly perform their desired tasks on a repeated basis and thereby develop (or drop) a habit. Various additional benefits will be apparent in view of the foregoing.

It should be apparent from the foregoing description that various example embodiments of the invention may be implemented in hardware or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A wearable device comprising:
A user interface for outputting communications to the user;
a band configured to at least partially surround a body part of a user;
a clasp configured to hold the band in a closed position, wherein the band resists removal from the body part while in the closed position;
a lock actuator configured to selectively lock the clasp against moving to an open position;
a communication interface configured to receive data from at least one other device; and
a processor in communication with the lock actuator and the communication interface, wherein the processor is configured to:
receive a communication indication that the wearable device is to output a specified communication to the user,
in response to receiving the communication indication, output the specified communication via the user interface,
receive a locking indication that the wearable device is to be unlocked, and
in response to receiving the locking indication, signal the lock actuator to allow the clasp to transition to the open position.

2. The wearable device of claim 1, wherein the locking indication is received via the communication interface.

3. The wearable device of claim 2, wherein the locking indication is received from a user device of a supporting user other than the user.

4. The wearable device of claim 1, further comprising: a memory configured to store a locking rule, wherein, in receiving the locking indication that the wearable device is to be unlocked, the processor is configured to evaluate the locking rule against a current context and, based on the evaluation, determine that the wearable device is to be unlocked.

5. The wearable device of claim 4, wherein, in evaluating the locking rule against a current context, the processor is configured to determine that the locking rule has expired and that, based on the expiration, the wearable device is to be unlocked.

6. The wearable device of claim 1, further comprising:
a sensor configured to obtain physiological data from the user when the band surrounds the body part,
wherein the processor is configured to transmit the physiological data via the communications interface.

7. (canceled)

8. The wearable device of claim 1, wherein:
the user interface comprises a vibrator; and
the specified communication is at least one vibration.

9. The wearable device of claim 1, further comprising:
a sensor configured to obtain physiological data from the user when the band surrounds the body part,
a memory configured to store a communication rule, wherein, in receiving the communication indication that the wearable device is to output a specified communication to the user, the processor is configured to evaluate the communication rule against a current context and, based on the evaluation, the wearable device is to output a specified communication to the user.

10. The wearable device of claim 9, further comprising:
a sensor configured to obtain physiological data from the user when the band surrounds the body part,
wherein, in evaluating the communication rule against a current context, the processor is configured to compare the communication rule to the obtained physiological data.

11. A locking rules engine comprising:
a communication interface configured to communicate with at least one other device;
a memory storing a user profile including an identification of a locking wearable device and a locking rule; and
a processor configured to:
evaluate the locking rule against a current context to determine whether the locking wearable device is to be unlocked; and
in response to determining that the locking wearable device is to be unlocked, transmit a locking instruction to the locking wearable device, wherein the locking instruction instructs the locking wearable device to unlock.

12. The locking rules engine of claim 11, wherein, in evaluating the locking rule against the current context, the processor is configured to determine when the locking rule is expired and that, consequently, the locking device is to be unlocked.

13. The locking rules engine of claim 11, wherein the processor is further configured to:
receive physiological data descriptive of the a user of the locking wearable device;
evaluate a goal previously received from the user against the received physiological data; and
based on the evaluation, transmit a communication instruction to the locking wearable device instructing the locking wearable device to output a specified communication to the user.

14. The locking rules engine of claim 13, wherein the processor is configured to transmit the communication instruction based on the evaluation indicating that the user has failed to meet the goal previously received from the user.

15. A system comprising the wearable device of claim 1 and the locking rules engine of claim 11.
